(19) United States
(12) Patent Application Publication
(10) Pub. No.: US 2008/0181928 A1
(43) Pub. Date: Jul. 31, 2008

(54) COATINGS FOR IMPLANTABLE MEDICAL DEVICES FOR LIPOSOME DELIVERY

(75) Inventors: Dorna Hakimi-Mehr, Vancouver (CA); Mark Landy, Atlanta, GA (US); Manus Tsui, Richmond (CA); Vlad Budzynski, North Vancouver (CA); Aleksy Tsetkov, Richmond (CA)

Correspondence Address: RISSMAN JOBSE HENDRICKS & OLIVERIO, LLP, 100 Cambridge Street, Suite 2101, BOSTON, MA, 02114, US

(73) Assignee: MIV Therapeutics, Inc., Vancouver (CA)

(21) Appl. No.: 11/960,974

(22) Filed: Dec. 20, 2007

Related U.S. Application Data
(60) Provisional application No. 60/876,829, filed on Dec. 22, 2006.
(60) Provisional application No. 60/942,565, filed on Jun. 7, 2007.
(60) Provisional application No. 60/981,245, filed on Oct. 19, 2007.

(51) Int. Cl. A61F 2/04; A61K 31/337; A61K 31/506; A61P 35/00; A61P 29/00; A61P 9/00

(52) U.S. Cl. 424/426; 424/423; 514/449; 514/252.18; 427/2.25; 205/317; 204/471

(57) ABSTRACT

Disclosed herein are medical devices, such as implantable medical devices (e.g., stents), comprising at least one coating covering at least a portion of the device comprising dry film. The dry film comprises at least one lipid bilayer and at least one pharmaceutically effective agent. Upon exposure to an aqueous fluid, liposomes are released comprising lipids from the dry film encapsulating the pharmaceutically effective agent. The film can contact the device directly or can be coated on a substrate, such as a ceramic.

### RELATED APPLICATIONS

This application claims the benefit of priority under 35 U.S.C. § 119(e) to U.S. Provisional Appl. No. 60/876,829, filed Dec. 22, 2006, U.S. Prov. App. No. 60/942,565, filed Jun. 7, 2007, and U.S. Prov. App. No. 60/981,245, filed Oct. 19, 2007, the disclosures of which are incorporated herein by reference.

### FIELD OF THE INVENTION

Disclosed herein are coatings for medical devices, such as implantable medical devices (e.g., stents), and processes for making the same. The coating comprises and a film containing a lipid bilayer and one or more therapeutic agents.

### BACKGROUND OF THE INVENTION

Implantable medical devices are used in a wide range of applications including bone and dental replacements and materials, vascular grafts, shunts and stents, and implants designed solely for prolonged release of drugs. The devices may be made of metals, alloys, polymers or ceramics.

Arterial stents have been used for many years to prevent restenosis after balloon angioplasty (expanding) of arteries narrowed by atherosclerosis or other conditions. Restenosis involves inflammation and the migration and proliferation of smooth muscle cells of the arterial media (the middle layer of the vessel wall) into the intima (the inner layer of the vessel wall) and lumen of the newly expanded vessel. This migration and proliferation is called neointima formation. The inflammation is at least partly related to the presence of macrophages. The macrophages are also known to secrete cytokines and other agents that stimulate the abnormal migration and proliferation of smooth muscle cells. Stents reduce but do not eliminate restenosis.

Drug eluting stents have been developed to elute anti-proliferative drugs from a non-degradable polymer coating and are currently used to further reduce the incidence of restenosis. Examples of such stents are the Cypher® stent, which elutes sirolimus, and the Taxus® stent, which elutes paclitaxel. Recently it has been found that both of these stents, though effective at preventing restenosis, cause potentially fatal thromboses (clots) months or years after implantation. Late stent thrombosis is thought to be due to the persistence of the somewhat toxic drug or the polymer coating or both on the stent for long time periods. Examination of some of these stents removed from patients frequently shows no covering of the stent by the vascular endothelial cells of the vessel intima. This is consistent with the possible toxicity of the retained drugs or non-degradable polymer. The lack of endothelialization may contribute to clot formation.

There have been attempts to develop polymer-free coatings. However, these approaches have failed to produce the desired outcomes due to problems such as lack of mechanical integrity necessary to undergo device preparation and implantation, and may also result in undesirably fast release of the therapeutic agent.

Accordingly, there remains a need to develop new drug eluting stents having sufficient efficacy, mechanical integrity, and a surface that is biocompatible.

### SUMMARY OF THE INVENTION

One embodiment provides a stent, comprising at least one coating covering at least a portion thereof, the at least one coating comprising a dry film comprising at least one lipid bilayer and at least one pharmaceutically effective agent.

Another embodiment provides a method of preparing a coating for a stent, comprising:

combining at least one lipid with at least one pharmaceutically active agent to form a composition comprising at least one lipid bilayer; and

coating at least a portion of the stent with the composition.

Another embodiment provides a method of preparing a coating for an implantable medical device, comprising:

coating at least a portion of the device with a substrate by at least one method selected from electrochemical deposition, electrophoretic deposition (EPD), sol gel processes, aero-sol gel processes, biomimetic (BM) processes, spraying, and dipping;

combining at least one lipid with at least one pharmaceutically active agent to form a composition comprising at least one lipid bilayer; and

coating at least a portion of the substrate with the composition.

Another embodiment provides a method of preparing a coating for an implantable medical device, comprising:

coating at least a portion of the device by depositing a substrate from a solution or suspension;

combining at least one lipid with at least one pharmaceutically active agent to form a composition comprising at least one lipid bilayer; and

coating at least a portion of the substrate with the composition.

Another embodiment provides a method of treating at least one disease or condition comprising:

implanting in a subject in need thereof a medical device comprising a coating covering at least a portion of the device, the coating comprising at least one lipid bilayer and a therapeutically effective amount of at least one pharmaceutically active agent, and

releasing from the device the at least one pharmaceutically active agent encapsulated in a liposome comprising lipids from the lipid bilayer.

Another embodiment provides a medical device comprising at least one coating covering at least a portion of the device, the at least one coating comprising:

a porous substrate;

a composition contacting the porous substrate, the composition comprising at least one lipid and at least one pharmaceutically effective agent wherein the at least one lipid does not form a lipid bilayer film; and

a dry lipid bilayer film contacting the porous substrate and/or the composition, the dry film comprising at least one pharmaceutically effective agent that can be the same or different from the agent in the composition.

Another embodiment provides a medical device comprising at least one coating covering at least a portion of the device, the at least one coating comprising:

a porous substrate;

a composition impregnating the porous substrate, the composition comprising at least one lipid and at least one pharmaceutically effective agent;

a film overcoating the composition, the film comprising at least one pharmaceutically effective agent and at least one lipid.

A medical device comprising at least one coating covering at least a portion of the device, the at least one coating comprising:

a porous substrate;

at least one pharmaceutically effective agent impregnating the porous substrate; and

a film overcoating the porous substrate, the film comprising at least one pharmaceutically effective agent and at least one lipid.

A medical device comprising at least one coating covering at least a portion of the device, the at least one coating comprising:

a porous substrate;

a film deposited on the substrate comprising at least one lipid and at least one pharmaceutically active agent; and

at least one pharmaceutically active agent contacting the porous substrate and free of contact with the film.

A method of treating at least one disease or condition comprising:

implanting in a subject in need thereof a medical device comprising at least one coating covering at least a portion of the device, the at least one coating comprising:
a substrate; and
a composition comprising at least one lipid and at least one pharmaceutically active agent, the composition covering at least a portion of the substrate; and

releasing from the device the least one pharmaceutically active agent encapsulated in the at least one lipid.

### BRIEF DESCRIPTION OF THE DRAWINGS

Various embodiments of the invention will be understood from the following description, the appended claims and the accompanying drawings, in which:

FIG. 1 is a schematic diagram showing the formation of liposomes from a dry lipid film;

FIGS. 2A-2C are optical micrographs of a stainless tube coated with Formulation B of Example 1 immediately upon immersion in PBS, (FIG. 2A), 30 minutes after immersion (FIG. 2B), and 60 minutes after immersion (FIG. 2C) at a magnification of approximately ×40;

FIG. 3 is a graph of the amount of liposome encapsulated paclitaxel released (y-axis) over time (x-axis) for Formulation A (♦) and Formulation B (▪) of Example 2;

FIGS. 4A and 4B are bar graphs of the amount of liposome encapsulated paclitaxel released versus free paclitaxel released (y-axis) at 1 h and 24 h (x-axis) for Formulation A (FIG. 4A) and Formulation B (FIG. 4B) of Example 2;

FIG. 5 is an optical micrograph of Formulation B of Example 2 after 1 h;

FIG. 6 are optical pictures of porous HAp coated stent (A) before and (B) after coating with a lipid formulation;

FIG. 7 is a graph of % cell growth inhibition (y-axis) versus cell treatment (x-axis) to indicate the inhibitory effect of hydroxyapatite-coated stents further coated with the ZA-containing bilayer formulation of Example 4, compared to molecular ZA added directly to THP-1 culture;

FIG. 8A is a schematic of porous HAp coating further coated with a lipid formulation containing ZA and (B) molecular ZA;

FIG. 8B is a schematic of porous HAp coating further coated with molecular ZA;

FIG. 9 is a graph of % cell growth inhibition (y-axis) versus cell treatment (x-axis) to indicate the inhibitory effect of stents coated with the lipid formulation of Example 4 on porous HAp versus a porous HAp coated stent impregnated with molecular ZA;

FIG. 10 is a schematic presentation of a dual drug coated stent coated with HAp where midostaurin is directly deposited in the pores of the hydroxyapatite coating, and the ZA lipid formulation is applied on top of this assembly;

FIG. 11 shows the elution profile of midostaurin (PKC-412) from the dual-drug stent of Example 9 as a plot of % PKC-412 cumulative release (y-axis) versus time (x-axis).

FIG. 12 is a schematic diagram of a device that can effect dual functionality of a single drug;

FIG. 13A is a plot of the amount of lipid and drug released (%, y-axis) from a stent of Example 14 over time (min., x-axis);

FIG. 13B is a plot of % weight loss of a lipid bilayer film (y-axis) over time (min., x-axis) from a stent of Example 14;

FIG. 14 is a plot of the total amount of PKC-412 released and the amount of encapsulated PKC-412 released (μg, y-axis) at 0, 15 and 60 minute intervals (x-axis) from a stent of Example 15;

FIGS. 15A and 15B are optical micrographs showing liposomes of various sizes formed and released from lipid-bilayer coated stents of Example 15;

FIG. 16 is a plot of % inhibition of growth of THP-1 cell growth (y-axis) for the zoledronic acid formulations (x-axis) of Example 16;

FIG. 17 is a plot of % inhibition of HCASMC growth (y-axis) for the midostaurin formulations of Example 17 at various time intervals (weeks, x-axis); and

FIG. 18 is a plot of % inhibition of THP-1 cell growth (y-axis) for the various midostaurin formulations (x-axis) of Example 18; and

FIG. 19 is a graph of % inhibition (y-axis) by the castor oil-midostaurin formulation of Example 20 at various time intervals (weeks, x-axis).

### DETAILED DESCRIPTION

One embodiment provides an implantable medical device, comprising a coating or film covering at least a portion of the device, where the coating or film comprises at least one lipid bilayer and a therapeutically effective amount of at least one pharmaceutically active agent. In one embodiment, the coating or film is capable of forming a liposome encapsulating the pharmaceutically active agent upon release of the agent from the device.

In one embodiment, the film is a dry film comprising at least one lipid bilayer and at least one pharmaceutically effective agent. Upon exposing the dry film to an aqueous solution, at least some of the agent is released as liposomes. In one embodiment, “dry film” refers to a film having a total amount of solvent (e.g., water and/or organic solvents) of less than 10%, such as a total amount of less than 5%, or a total amount of solvent of less than 2%, or even a total amount of solvent less than 1%.

“Lipid bilayer” as used herein refers to a structure formed by amphipathic (containing both hydrophilic and hydrophobic groups) lipids. Such lipids have polar head groups and nonpolar tails. In an aqueous medium, they align in two layers where the hydrocarbon tails of one monolayer face the tails of a second monolayer to form a nonaqueous inner portion, e.g., a bilayer membrane. The polar heads line the periphery of the bilayer to face the aqueous medium. In one embodiment, a “lipid bilayer” refers to at least one continuous sheet of a bilayer membrane, as opposed to comprising predominantly closed vesicles. “At least one lipid bilayer” as used herein refers to single or multiple (two or more) layers of bilayers.

In one embodiment, “aqueous solution” refers to an in vitro solution comprising water and optionally including buffers and/or other components, such as those components that adjust the solution to a desired pH. In another embodiment, the aqueous solution is a body fluid.

In one embodiment, the lipid bilayer in the dry film is capable of forming liposomes. Providing a device with lipid bilayers allows the simple preparation of a dry lipid film such that when the film contacts a physiological or aqueous medium, the film absorbs water, swells, and the at least one pharmaceutically active agent is released from the device encapsulated in a liposome without performing the extra steps of preforming the liposome. “Liposomes” as used herein refers to closed vesicles (e.g., substantially spherical vesicles) formed under osmotically balanced conditions comprising molecular bilayers of amphiphiles having their hydrophobic portions forming the interior of the bilayer and their hydrophilic portions contacting an aqueous phase. Liposomes can have one or more bilayers.

The phospholipids and/or other amphiphiles that form liposomes may be used to release agents or to target agents to particular cells or organs. Liposomes can serve to protect agents from degradation by shielding them from catabolic enzymes and by prolonging their circulation time in the blood.

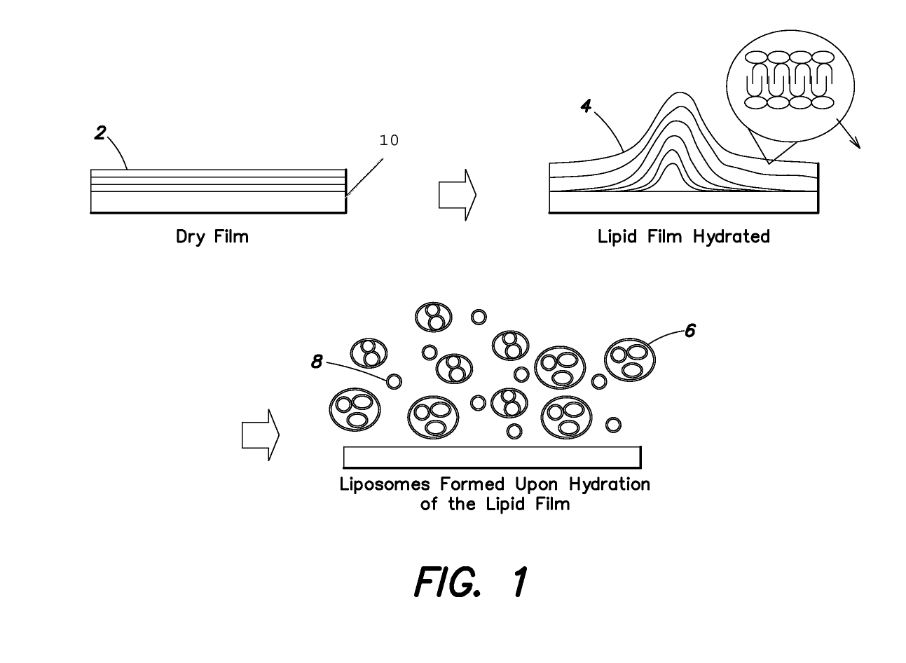

As schematically depicted in FIG. 1, as a lipid-containing film 2 on a surface 10 is exposed to a physiological medium, water or other aqueous solutions (e.g., buffer solution), water is absorbed by the film to cause swelling of the layers. Hydrated film 4 maintains the lipid film 2 until the lipids are released to form multi-vesicular 6 or unilamellar liposomes 8 encapsulating the pharmaceutically active agent. Multi-lamellar liposomes (not shown) can be formed as well.

Previous studies have prepared stents coated with preformed liposomes encapsulating a drug with the intent of using the liposomes merely as a drug repository for sustained release of free drug (e.g., unencapsulated by a liposome). In one embodiment, the dry film comprising a bilayer can release a greater amount of liposome encapsulated drug than a coating comprising a preformed liposome. The use of a dry film disclosed herein (as opposed to preformed liposomes) can also provide the added benefit of simplified manufacture of a liposome delivery coating without performing the extra steps of preforming a liposome to be coated on a stent.

The pharmaceutically active agent in the bilayer film can be hydrophilic, hydrophobic, or amphipathic. In one embodiment, the agent is contained in the hydrophobic phospholipid tail region of a membrane bilayer. In another embodiment the agent is contained in the hydrophilic head group region of a membrane bilayer. When released from the bilayer, the agent can be contained in the interior aqueous compartment of a liposome, or can be present in the nonpolar tail region of the liposome.

When implanted in the body, drug-coated devices typically exhibit an initial “burst release” in which an excessive amount of drug is released from the device. This burst release can render ineffective sustained drug delivery. Liposomes can provide a matrix to deliver therapeutic agents (e.g., hydrophilic drugs) to a target tissue and reduce burst release. Encapsulating a hydrophilic drug within the liposomal bilayer membrane can reduce or even prevent premature washout of a water soluble drug from the tissue. Because of the increased residence time of the drug, a treatment regimen involving liposome encapsulated agents may allow a reduced dosage. In the case of hydrophobic drugs, a liposome can improve the solubility of a drug and control its release from the coating of the device.

Liposomes can comprise one or more lipid types. The type of lipid and their relative ratios can be tailored to effect a burst release, prevent a burst release, or otherwise control the length of time of sustained delivery of a pharmaceutically active agent. The lipids can be chosen depending on the hydrophilicity or hydrophobicity of the drug to improve the solubility of a hydrophobic drug or prevent premature washout of a hydrophilic drug. Exemplary lipids are disclosed in further detail below.

Liposomes can also be used to target agents to macrophages due to the high rate of phagocytosis by these cells. In general macrophages preferentially take up larger liposomes (1-2 μm, Chono et al 2006), liposomes with negatively charged phospholipids (Fidler, 1988; Lee et al, 1992) and, in general, liposomes with a more fluid membrane (Allen et al, 1991). For some liposomes, increasing cholesterol content can increase overall uptake by macrophages even though the cholesterol may cause decreased membrane fluidity (Huong et al 1998).

In some instances, macrophages can take up certain particles having a diameter of about 1-2 μm or greater. Liposomes can be designed to have a diameter ranging from of about 1-2 μm and greater in order to increase their uptake by macrophages and reduce inflammation, such as the inflammation component of restenosis. In one embodiment the lipid bilayer film releases therapeutic agent-containing liposomes having a diameter of about 1-2 μm or greater to inhibit macrophages and prevent inflammation. In one embodiment, at least 5%, at least 10% or at least 25% of the drug is released as liposomes having a diameter of about 1-2 μm or greater.

In one embodiment, a first population of the drug in the bilayer film is released as liposomes and a second population of the drug in the bilayer film is released as free drug. This embodiment releases the drug in two different forms and can enable the drug to exhibit dual functionality: (1) the drug released in liposomes having a diameter of greater than 1 or 2 μm can be taken up by macrophages to treat a first condition, such as an inflammatory reaction, and (2) the same drug in free form can treat a second condition, e.g., proliferation. In one embodiment, for the treatment of restenosis, a drug known for being an antiproliferative agent can be released encapsulated in a liposome to reduce the number of inflammatory agents whereas the free form of the drug can act to inhibit proliferation of smooth muscle cells, e.g., a drug delivered in liposome and free form can have both anti-inflammatory and antiproliferative activity due to the dual delivery form and potentially eliminating the need to deliver a separate anti-inflammatory drug.

In one embodiment, the liposomes released from the device have a variety of particles sizes. In one embodiment, the liposomes exhibit a particle size distribution, wherein at least 5%, or at least 10% of particles released as liposomes have an average diameter of less than 1 μm, and at least 25%, or at least 50% of the particles released as liposomes have an average diameter of greater than 1 μm. In another embodiment, less than 25% of the particles released as liposomes have an average diameter of less than 1 μm, or less than 10% of the particles released as liposomes have an average diameter of less than 1 μm. In another embodiment, at least 10% of the particles released as liposomes have an average diameter of greater than 2 μm, or at least 25%. of the particles released as liposomes have an average diameter of greater than 2 μm.

Depending on the treatment and/or pharmaceutically active agent (e.g., a bisphosphonate and optionally other therapeutic agent), a certain ratio of agent released in a liposome compared to that released as free agent may be desired. Tailoring the film composition (e.g., concentrations and type of lipid and/or pharmaceutically active agent) can, in one embodiment, alter the amount of agent released encapsulated in a liposome relative to the amount of free (unencapsulated) agent released. In one embodiment, the amount of agent released encapsulated in a liposome is at least 10%, relative to the total amount of agent initially in the dry film. In another embodiment, the amount of agent released encapsulated in a liposome is at least 25%, at least 50%, or at least 75%, relative to the total amount of agent initially in the dry film. In another embodiment, the amount of agent released encapsulated in a liposome is no more than 25%, such as an amount of no more than 50% or no more than 75%, relative to the total amount of agent initially in the dry film.

### The Lipid Bilayer

The lipids forming the lipid bilayer can be selected from a number of lipids such as phospholipids and glycolipids. Alternatively, in one embodiment, the bilayer can comprise lipids other than phospholipids or glycolipids, including sphingomyelins, cerebrosides, ceramides, gangliosides, and sulfatides. In another embodiment, these lipids can be present in the bilayer in addition to the phospholipid or glycolipid.

In one embodiment, the lipids can have two identical fatty acid chains. The fatty acids can comprise C₄-C₃₂ hydrocarbon chains, such as C₈-C₂₈ hydrocarbon chains, C₆-C₂₄ hydrocarbon chains, C₁₂-C₃₂ hydrocarbon chains, or even C₁₂-C₂₄ hydrocarbon chains. In another embodiment, the lipids can be identical or different, saturated or unsaturated (e.g., containing up to 6 double bonds in cis or trans configurations). The bilayer can comprise one or more of the lipid types disclosed herein.

Exemplary phospholipids include phosphoglycerides, such as phosphatidylcholines, phosphatidylethanolamines, phosphatidylglycerols (e.g., cardiolipin), phosphatidic acids, phosphatidylserines, and phosphatidylinositols.

Exemplary phosphatidylcholines include those selected from 1,2-dilauroyl-sn-glycero-3-phosphocholine, 1,2-dimyristoyl-sn-glycero-3-phosphocholine, 1,2-dipalmitoyl-sn-glycero-3-phosphocholine, 1,2-distearoyl-sn-glycero-3-phosphocholine, 1,2-dioleoyl-sn-glycero-3-phosphcholine, 1-palmitoyl-2-oleoyl-sn-glycero-3-phosphocholine, egg phosphatidylcholine, hydrogenated egg phosphatidylcholine, soybean phosphatidylcholine, and hydrogenated soybean phosphatidylcholine.

Exemplary phosphatidylethanolamines include those selected from 1,2-dimyristoyl-sn-glycero-3-phosphoethanolamine, 1,2-dipalmitoyl-sn-glycero-3-phosphoethanolamine, 1,2-distearoyl-sn-glycero-3-phosphoethanolamine, and 1,2-dioleoyl-sn-glycero-3-phosphoethanolamine.

Exemplary phosphatidylglycerols include those selected from egg phosphatidylglycerol, 1,2-dimyristoyl-sn-glycero-3-phosphoglycerol, 1,2-dipalmitoyl-sn-glycero-3-phosphoglycerol, 1,2-distearoyl-sn-glycero-3-phosphoglycerol, and 1-palmitoyl-2-oleoyl-sn-glycero-3-phosphoglycerol.

Exemplary phosphatidic acids are selected from 1,2-dimyristoyl-sn-glycero-3-phosphate, 1,2-dipalmitoyl-sn-glycero-3-phosphate, and 1,2-distearol-sn-glycero-3-phosphate.

In one embodiment, the phospholipid is the bilayer forming component. Other lipids can be added to tailor the properties of the bilayer, e.g., mechanical rigidity or crystallinity, fluidity, etc. In one embodiment, the phospholipid-containing bilayer further comprises glycolipids. Exemplary glycolipids include glucosyl, galactosyl, lactosyl ceramide, ceramide, phosphocholine ceramide, sulfogalactosyl ceramide, cerebrosides, sulfolipids (e.g., sulfatide), and gangliosides.

In one embodiment, where a glycolipid is the bilayer forming component, the glycolipid is chosen from those glycolipids capable of forming a bilayer.

In one embodiment, the dry film further comprises additional components, whether or not they are capable of forming a bilayer, so long as these components are added in an amount so as not to disrupt the overall bilayer structure. In one embodiment, the dry film further comprises cholesterol and/or cholesterol derivatives. Cholesterol has a structure well known in the art with a weakly polar hydroxyl group on the A ring of its rigid four-fused ring system and a short hydrocarbon tail on the D ring at the other end of the molecule.

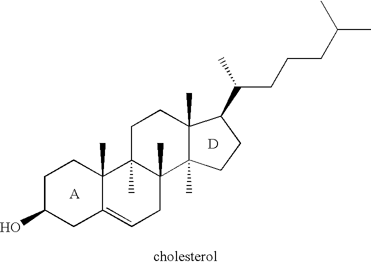

Cholesterol may be capable of aligning in a lipid bilayer where its hydroxyl group is oriented towards the polar head-group region and its rings and tail are oriented towards the interior hydrocarbon region (D. Voet and J. G. Voet, Biochemistry, Second Edition, John Wiley, New York, 1995).

In one embodiment, cholesterol and/or derivatives thereof are added in an amount sufficient to impart greater rigidity and/or stability to the bilayer film. The lipids forming the bilayer film typically have a more flexible structure compared to cholesterol. Cholesterol with its fused ring structure has molecular rigidity that it can impart to the bilayer structure. In another embodiment, the cholesterol can be present in an amount sufficient to impart a desired rigidity to the bilayer. In another embodiment, cholesterol is present to improve long term blood compatibility of the dry film.

“Cholesterol derivatives” as used herein refer to those compounds that mimic the alignment of cholesterol in a lipid bilayer. In one embodiment, cholesterol derivatives have the same four-fused ring system as cholesterol, with at least one weakly polar group on the A ring and a short hydrocarbon tail on the D ring. In one embodiment, the hydrocarbon tail is a C₂-C₁₁, branched or straight chain, saturated or unsaturated (e.g., 1, 2, or 3 double bonds) hydrocarbon.

Exemplary cholesterol derivatives include 7β-hydroxycholesterol 7-ketocholesterol, 7-ketocholesteryl acetate, 25-hydroxycholesterol, 24,25-epoxycholesterol, diacetylenic cholesterol, cholest-4-ene-3,6-dione, cholest-4-en-3-one, cholesteryl behenate, cholesteryl benzoate, cholesteryl butyrate, cholesteryl caprate, cholesteryl caproate, cholesteryl caprylate, cholesteryl-3,5-dinitrobenzoate, cholesteryl formate, cholesteryl-β-D-glucoside, cholesteryl hemisuccinate, cholesteryl heptylate, cholesteryl heptadecanoate, cholesteryl hydrogen phthalate, cholesteryl isobutyrate, cholesteryl isovalerate, cholesteryl laurate, cholesteryl linoleate, cholesteryl methyl succinates, cholesteryl myristate, cholesteryl nervonate, cholesteryl-p-nitrobenzoate, cholesteryl oleate, cholesteryl oleyl carbonate, cholesteryl palmitate, cholesteryl palmitelaidate, cholesteryl palmitoleate, cholesteryl phosphoryl choline, cholesteryl polyethylene glycols, cholesteryl propionate, cholesteryl N-propyl carbonate, cholesteryl 1-pyreecarbonate, cholesteryl (pyren-1-yl) hexanoate, cholesteryl stearate, cholesteryl-P-tosylate, cholesteryl valerate, thiocholesterol, and cholesteryl sulfate.

Other exemplary cholesterol derivatives include lanosterol, 14-nor-lanosterol, 14-nor,24,25-dihydrolanosterol, Δ⁷-cholestenol, 4α-methyl-Δ⁷-cholestenol, 4α-methyl-Δ⁸-cholestenol, dehydrocholesterol, cholestenone, cholestanone, cholestanol, coprosterol (coprostanol), coprostanone, 7α-hydroxycholesterol, 7αhydroxy-4-cholesten-3-one, 5β-cholestan-3α,7α,12α,26-tetrol, 7α,12α-dihydroxy-4-cholesten-3-one, 5β-cholestan-3α,7α,12α-triol, 5β-cholestan-3α,7α-diol, 5β-cholestan-3α,7α,26-triol, 5-cholestene-3β,7β-diol, 5-cholestene-3β,20α-diol, 5-cholestene-3β,22(R)-diol, 5-cholestene-3β,22(S)-diol, 5-cholestene-3β,25-diol, 5α-choles-7-en-3β-ol, 5α-choles-3β-ol-7one, 5α-cholestan-3β-ol, 5β-cholestan-3α-ol, α1-sitosterol, β-sitosterol, γ-sitosterol, stigmasterol, stigmastanol, fucosterol, campesterol, ergostanol, α-ergostenol, β-ergostenol, γ-ergostenol, dinosterol, and ergosterol.

### Methods of Preparing the Dry Film

In one embodiment, the dry film is prepared by combining at least one lipid with at least one pharmaceutically active agent to form a composition comprising at least one lipid bilayer. In one embodiment, the combining comprises forming a solution, suspension, or emulsion containing the lipid(s) and agent(s) followed by coating onto the device by any number of methods.

In one embodiment, the combining comprises forming a homogeneous solution comprising the at least one lipid and at least one pharmaceutically active agent. In one embodiment, the at least one pharmaceutically active agent is hydrophobic or amphipathic. A hydrophobic agent generally dissolves more readily in oils or non-polar solvents than in water, but may have some solubility in water. For somewhat hydrophobic, hydrophilic, or amphipathic agents, one of ordinary skill in the art can determine through experimentation whether to use the solution or emulsion method.

In one embodiment, the combining comprises forming a water-in-oil emulsion. Any type of pharmaceutically active agent can be used in this method. In one embodiment, the water-in-oil emulsion comprises at least one hydrophilic (e.g., dissolves more readily in water than in oils or non-polar solvents) or amphipathic pharmaceutically active agent. In one embodiment, the at least one lipid is dissolved in an organic solvent immiscible with water, e.g., one or more low boiling point organic solvents such as dichloromethane, diethyl ether, and chloroform. The at least one pharmaceutically active agent can be dissolved in an aqueous medium and combined with the lipid-containing solution to form a water-in-oil emulsion, where the polar, hydrophilic head of the lipid has a higher affinity for the water droplet and the hydrophilic tails remain in the organic phase to encapsulate the aqueous solution containing the pharmaceutically active agent.

Various techniques are known in the art for forming a stable microemulsion having a desired droplet size. In one embodiment, the droplet size ranges from 1 μm to 50 μm, such as a size ranging from 0.01 μm to 0.5 μm, to achieve a stable microemulsion. In one embodiment, at least one additional surfactant can be added to aid in forming the emulsion and/or stabilizing the emulsion to ensure a homogenous dispersion of the emulsified phase. The at least one additional surfactant can be ionic, such as those selected from chitosan, didodecyldimethylammonium bromide, and dextran salts, e.g., naturally occurring ionizable dextrans such as dextran sulfate or dextrans synthetically modified to contain ionizable functional groups. Exemplary nonionic surfactants include dextrans, polyoxyethylene castor oil, polyoxyethylene 35 soybean glycerides, glyceryl monooleate, triglyceryl monoleate, glyceryl monocaprylate, glycerol monocaprylocaprate, propylene glycol monolaurate, triglycerol monooleate, stearic glycerides, sorbitan monostearate (Span® 60), sorbitan monooleate (Span® 80), polyoxyethylene sorbitan monolaurate (Tween® 20), polyoxyethylenesorbitan tristearate (Tween® 65), and polyoxyethylene sorbitan monooleate (Tween® 80).

In one embodiment, binders (e.g., non-liposome forming materials such as hydrogenated vegetable oils) can be added to organic solution prior to forming the emulsion.

In one embodiment, the at least one pharmaceutically acceptable agent is distributed throughout the dry film, as opposed to having high and low areas of concentration in certain portions of the film.

In one embodiment, the at least one pharmaceutically acceptable agent is distributed within the lipid bilayer of the dry film. For example, a hydrophilic drug can be mainly distributed in the polar (head) region of the bilayer(s) whereas a hydrophobic drug can be mainly distributed in the non-polar (tail) region of the bilayer(s). An amphipathic drug can be distributed in both regions or be aligned in the bilayer similarly to the amphipathic lipids and may even be uniformly distributed throughout the bilayer-containing film.

### Substrates

In one embodiment, the device comprises an additional coating that serves as a substrate for the at least one lipid coating. The additional coating, or substrate, contacts the bilayer film and may directly contact the device, e.g., function as an inner coating.

The substrate can comprise one or more polymers typically used for implantable medical devices, as disclosed above. In other embodiments, the inner coating can be a ceramic, such as those ceramics known in the art to be biocompatible, e.g., hydroxyapatite, titanium oxide, and silicon carbide. Exemplary treatments/coatings of a surface with a ceramic material that improves the performance of subsequently deposited polymer layer is disclosed in WO 2006/024125, the disclosure of which is incorporated herein by reference. Alternatively, the inner coating can be an inorganic coating, such as metals (e.g. gold), or carbon.

In one embodiment, the substrate can comprise a ceramic. In certain embodiments of the invention where a ceramic substrate coats a medical device having a metallic surface, the drug may exhibit a greater binding affinity for the ceramic compared to the metal, thus slowing its release from the device when compared to a coating on the metal surface. Where the drug preferentially binds the ceramic, a liposome can effect or increase the rate of release of the drug from the device by providing a matrix for a drug. This mechanism may be useful in the situation where an increased rate of release of a drug is desired, e.g., and anti-inflammatory agent for treating the initial pathogenic activities in response to the implantation of the device.

In another embodiment, the substrate is porous. In one embodiment, the porous substrate can have pores and voids sufficiently large enough to contain a drug yet have passageways that permit the drug to be released from the pores of the substrate and enter the aqueous solution. In this embodiment, a porous substrate is provided that can act as a drug reservoir. The size and volume fraction of the substrate porosity can also be adjusted to influence the release rate of the therapeutic agent, e.g., by adjusting the porosity volume and/or pore diameter. For example a porous substrate possessing nano-size porosity is expected to decrease the release rate of the therapeutic agent compared to a porous substrate having micro-size porosity.

In one embodiment, the substrate is porous and has a porosity volume ranging from 30 to 70% and an average pore diameter ranging from 0.3 μm to 0.6 μm. In other embodiments, the porosity volume ranges from 30 to 60%, from 40 to 60%, from 30 to 50%, or from 40 to 50%, or even a porosity volume of 50%. In yet another embodiment, the average pore diameter ranges from 0.4 to 0.6 μm, from 0.3 to 0.5 μm, from 0.4 to 0.5 μm, or the average pore diameter can be 0.5 μm. For example, calcium phosphates displaying various combinations of the disclosed thicknesses, porosity volumes or average pore diameters can also be prepared.

Where the substrate is porous, the dry film can be layered on top of the porous surface. Alternatively, a lipid film can penetrate or impregnate the pores of the substrate, either throughout the entire depth of the substrate (wholly) or partially through the substrate. “Partial” impregnation can refer to a lipid film that impregnates only a portion of the porous substrate. In one embodiment, only the upper (or exposed) portion of the substrate is impregnated with the lipid film, where the lipid film does not impregnate the entire depth of the substrate. In another embodiment, the lipid film can partially impregnate only lower portion of the substrate, leaving the upper (exposed) portion of the porous substrate free of the film. In yet another embodiment, the lipid film uniformly partially impregnates the entire depth of the porous substrate.

In another embodiment, the lipid film can coat and contact the device, and the substrate can be deposited on top of the lipid film. Various layering embodiments of porous/nonporous substrates and lipid bilayer films can also be formed to create unique modes for drug delivery. One embodiment provides a medical device comprising at least one coating covering at least a portion of the device, the at least one coating comprising combinations of two or more of: (a) a nonporous substrate; (b) a porous substrate containing no lipid film or pharmaceutically active agents; (c) a porous substrate impregnated partially or wholly with only a lipid film; (d) a porous substrate impregnated partially or wholly with only pharmaceutically active agents; (e) a porous substrate impregnated partially or wholly with both a lipid film and pharmaceutically active agents; (f) a lipid film that is not a lipid bilayer film; and (g) a lipid bilayer film. The lipid film impregnating the porous substrate in (c) and (e) can be either a bilayer or nonbilayer film.

In one embodiment, a porous substrate may offer an opportunity for a single drug type to exhibit dual functionality. In conjunction with a drug impregnating the porous substrate, a film comprising a lipid bilayer and at least one pharmaceutically active agent can coat a top surface of the substrate. Accordingly, one embodiment provides a medical device comprising at least one coating covering at least a portion of the device, the at least one coating comprising:

a porous substrate;

a composition contacting the porous substrate, the composition comprising at least one lipid and at least one pharmaceutically effective agent wherein the at least one lipid does not form a lipid bilayer film; and

a dry lipid bilayer film contacting the porous substrate and/or the composition, the dry film comprising at least one pharmaceutically effective agent that can be the same or different from the agent in the composition.

Another embodiment provides embodiment provides a medical device comprising at least one coating covering at least a portion of the device, the at least one coating comprising:

a porous substrate;

a composition impregnating the porous substrate, the composition comprising at least one lipid and at least one pharmaceutically effective agent;

a film overcoating the composition, the film comprising at least one pharmaceutically effective agent and at least one lipid.

Another embodiment provides a medical device comprising at least one coating covering at least a portion of the device, the at least one coating comprising:

a porous substrate;

at least one pharmaceutically effective agent impregnating the porous substrate; and

a film overcoating the porous substrate, the film comprising at least one pharmaceutically effective agent and at least one lipid.

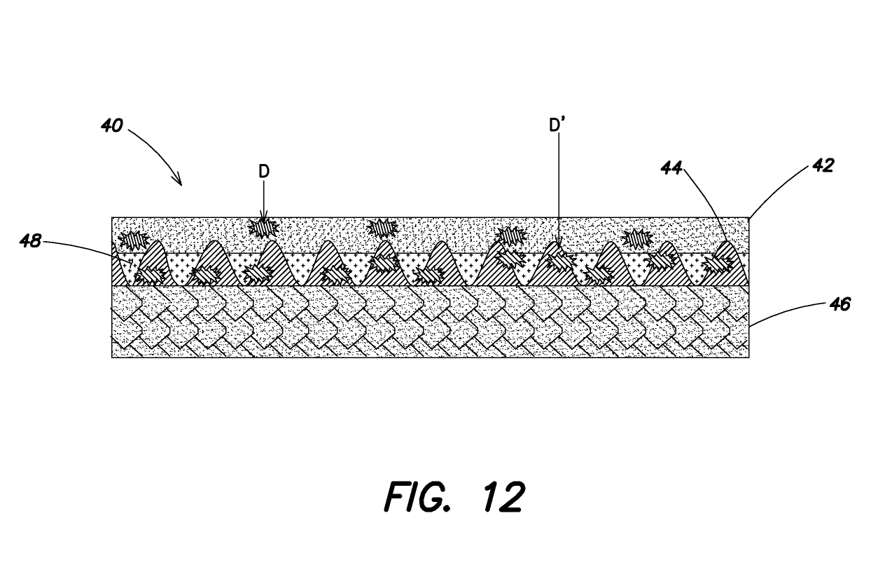

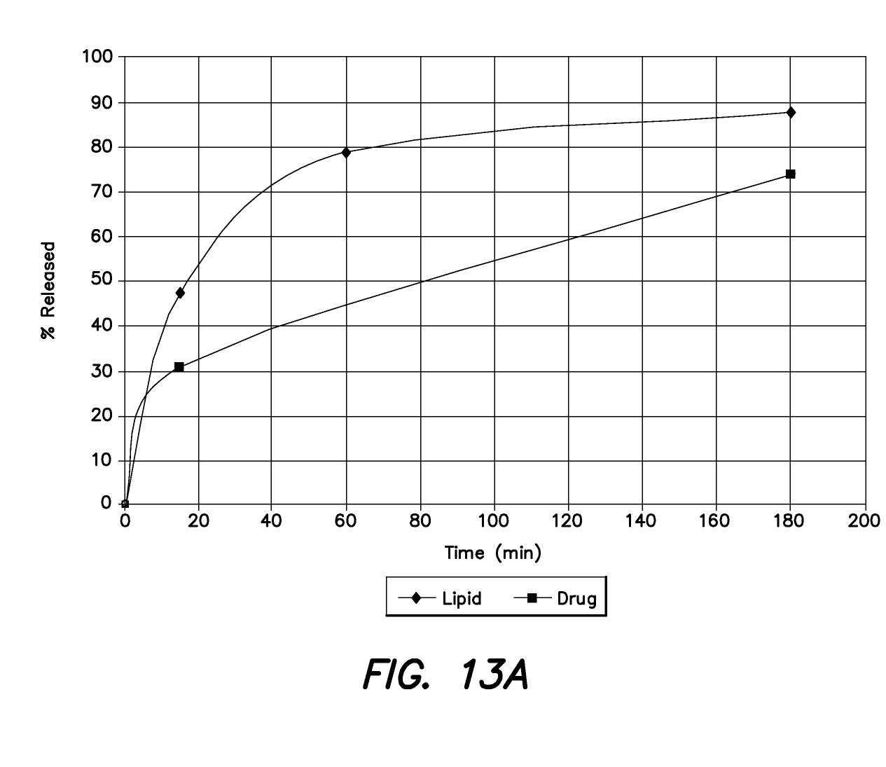

In one embodiment, a pharmaceutically active agent can impregnate a porous substrate and can be present in a lipid film that coats the substrate. FIG. 12 schematically shows an embodiment of a device 40 capable of delivering a single drug type having dual functionality. Device 40 comprises a surface 46 coated with a porous substrate 44, which can be a ceramic. Substrate 44 is further coated with a film 42 comprising at least one lipid, e.g., in the form of a lipid bilayer. The film 42 further comprises a drug D, which, when exposed to an aqueous solution can be released as a drug-encapsulated liposome (e.g., as illustrated schematically in FIG. 1). A drug D′, which can be the same or different from drug D, can be deposited in the pores of substrate 44. In one embodiment, at least some of the drug D′ is free of contact with the film 42.

Device 40 can operate in the following manner. Upon exposure to an aqueous solution, the drug D in film 42 can be released as a drug-encapsulated liposome. After, or simultaneously with, the consumption of film 42, drug D′ can exit the pores of substrate 44 through a network of cavities and voids and be released into the body. This dual drug delivery mode can be useful, e.g., in the treatment of one or more conditions (e.g., restenosis) in a manner that controls the order of release of the drug. In one embodiment, drug D is initially released as larger particles (in a liposome) for consumption by the macrophages in the treatment of inflammation. This drug delivery course can over a time period of less than 7 days, e.g., a time period of less than 3 days or less than 2 days. Drug D′ can be more slowly released from the porous substrate, depending on the porosity volume and pore size, to act as an antiproliferative agent over a longer course of time, e.g., at least 7 days, or at least 10 days and even up to a period of 1 year. In another embodiment, at least 50% of the drug D′ is released from the porous substrate over a period ranging from 7 days to 6 months, from 7 days to 3 months, from 7 days to 2 months, from 7 days to 1 month, from 10 days to 1 year, from 10 days to 6 months, from 10 days to 2 months, or from 10 days to 1 month.

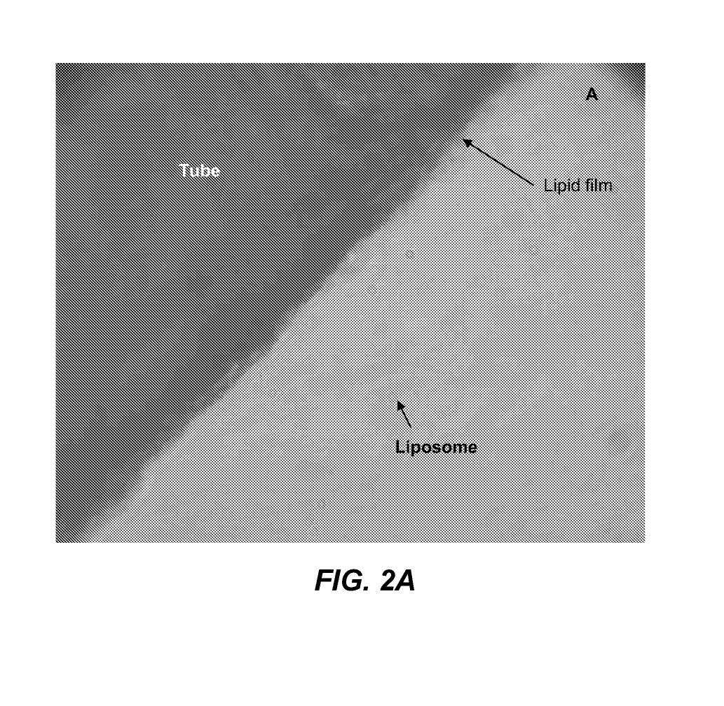

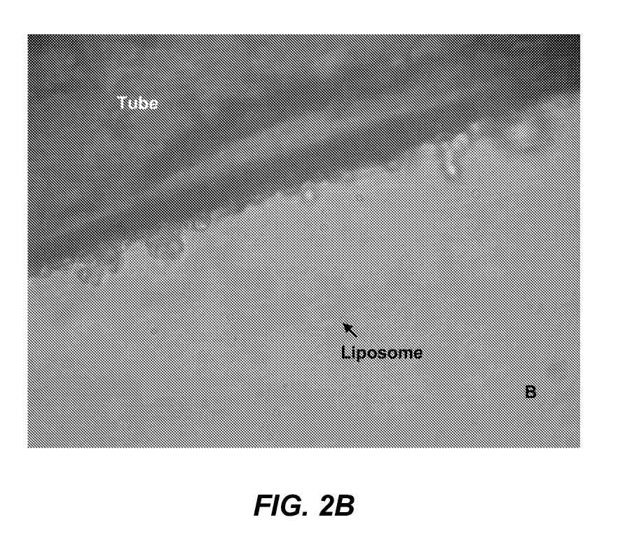

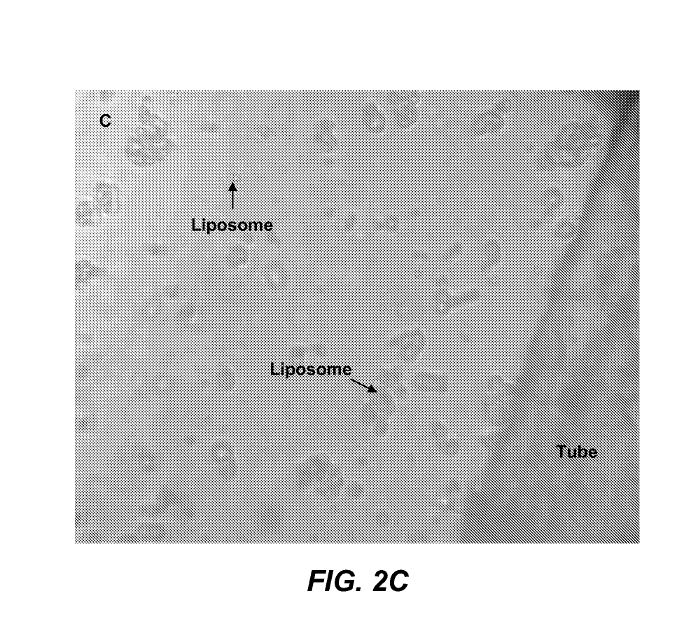

In one embodiment, the drug D′ is present in the pores in molecular or particulate form and is released from the substrate upon exposure to an aqueous solution. Some of drug D′ can be released in free form and/or encapsulated in a liposome. In another embodiment, the drug D′ is present in the pores with a biodegradable, pliable organic vehicle (e.g. vehicle 48 in FIG. 2). The organic vehicle my comprise a naturally occurring lipid such as a fat, oil, fatty acid, cholesterol, phospholipid or other lipid. In one embodiment, the lipid does not form liposomes. Biological lipids can provide a biodegradable and/or biocompatible vehicle for therapeutic agents. These lipids may include fats, oils, fatty acids, phospholipids and others. The fats, oils and fatty acids form a nearly water-insoluble vehicle which can release an agent by slow dissolution or biodegradation. Thus, the fat, oil, cholesterol or fatty acid vehicle can serve to control the release of a therapeutic agent contained therein by its biodegradation, slow dissolution, or slow release of the agent. The lipid can also help control the release of drug by retarding or increasing the rate of release depending on the relative miscibility of the lipid and drug. In another embodiment, the drug can be released from the porous substrate with the lipid as drug-encapsulated capsules (nanocapsules, microcapsules), droplets (nanodroplets, microdroplets), spheres (microspheres, nanospheres), and/or micelles. Such drug-encapsulated species may enhance the uptake of the therapeutic agent by the cells, improve the potency of the drug, and/or increase the residence time of the drug in the surrounding tissue by reducing the solubility of the therapeutic agent in the physiological fluids.

In another embodiment, the organic vehicle may comprise a polymer. Any polymer can be used, such as those polymers useful for preparing medical devices, e.g., the polymers listed in the “Devices” section below.

Another embodiment provides a medical device comprising at least one coating covering at least a portion of the device, the at least one coating comprising:

a first porous substrate contacting the device;

a lipid bilayer film overcoating the first porous substrate, the film comprising at least one pharmaceutically effective agent and at least one lipid; and

a second porous substrate coating the lipid bilayer film.

The agent in the porous substrate can be hydrophilic, hydrophobic, or amphipathic. In one embodiment the agent impregnating the porous substrate is soluble in the pliable vehicle. In another embodiment the agent is insoluble in the vehicle.

In one embodiment, the at least one pharmaceutically effective agent in the porous substrate acts primarily as an anti-proliferative agent and the agent in the film (e.g., the dry bilayer film) coating the substrate acts primarily as an anti-inflammatory agent. In one embodiment, the agents can inherently possess anti-proliferative and anti-inflammatory properties, respectively, such that they act primarily as respective anti-proliferative and anti-inflammatory agents, e.g., the drug in the porous substrate is different from the drug in the film. In another embodiment, the agent in the substrate and the film can be the same yet act primarily as respective anti-proliferative and anti-inflammatory agents. This can be possible because the agent, e.g., inherently an anti-proliferative agent, can be released from the film encapsulated in a liposome so as to have a size sufficient to be a target of macrophages. The same agent in the porous substrate, whether loaded in molecular form or in an organic vehicle, can be released in free form and act as primarily as an anti-proliferative agent, which is its inherent function.

In one embodiment, the pharmaceutically effective agent impregnating the porous substrate (e.g., drug D′ of FIG. 2) is different from the agent present in the film. This has been demonstrated in Example 9, where midostaurin impregnates the pores of the substrate and zoledronic acid is present in the lipid bilayer film. In another embodiment, more than one drug type can be present in the lipid bilayer film and/or can impregnate the pores of the porous substrate with or without a vehicle.

In one embodiment, the substrate, e.g., a ceramic, is biocompatible so as to provide a surface that can promote growth of endothelial cells of the vascular intima, i.e., endothelialization. Previously, drug eluting stents have been developed to elute anti-proliferative drugs from a non-degradable aromatic polymer coating and are currently used to further reduce the incidence of restenosis. Commercially available drug eluting stents, such as the Cypher® stent, which elutes sirolimus, and the Taxus® stent, which elutes paclitaxel, do not promote endothelialization, most likely because of the non-degradable polymer.

In one embodiment, upon resorption of the lipid bilayer film by the aqueous solution or body fluid, the surface of the biocompatible ceramic is exposed to the body fluid. Ceramics can persist in the body for one or more years, and a stable, persistent coating is not undesirable in the body since endothelialization has been demonstrated on biocompatible ceramics, such as a hydroxyapatite coating.

In one embodiment, the thickness of the porous substrate coating can be adjusted so that it provides the necessary volume for deposition of the composition comprising one or more lipids and one or more pharmaceutically active agents. In one embodiment, the adhesion of the porous substrate coating to the surface of the medical device is such that the porous substrate does not delaminate from the surface of the medical device during implantation. In one embodiment, the porous substrate has a thickness of 10 μm or less. In other embodiments, e.g., where the device is an orthopedic implant, the porous substrate can have a thickness ranging from 10 μm to 5 mm, such as a thickness ranging from 100 μm to 1 mm.

In one embodiment, the substrate is well bonded to the stent surface and neither forms significant cracks nor flakes off the stent during mounting on a balloon catheter and placement in an artery by expansion. In one embodiment, a substrate that does not form significant cracks can have still present minor crack formation so long as it measures less than 300 nm, such as cracks less than 200 nm, or even less than 100 nm.

In one embodiment, the substrate is a ceramic, such as any ceramic known in the art to be biocompatible, e.g., metal oxides such as titanium oxide, aluminum oxide, silica, and indium oxide, metal carbides such as silicon carbide, and one or more calcium phosphates such as hydroxyapatite, octacalcium phosphate, α- and β-tricalcium phosphates, amorphous calcium phosphate, dicalcium phosphate, calcium deficient hydroxyapatite, and tetracalcium phosphate.

In one embodiment, the substrate is a calcium phosphate coating, such as hydroxyapatite. The calcium phosphate coating may be deposited by electrochemical deposition (ECD) or electrophoretic deposition (EPD). In another embodiment the coating may be deposited by a sol gel (SG) or an aero-sol gel (ASG) process. In another embodiment the coating may be deposited by a biomimetic (BM) process. In another embodiment the coating may be deposited by a calcium phosphate cement (CPC) process. In another embodiment the coating may be deposited by a plasma deposition process, e.g., a plasma spray.

In one embodiment, the inner coating comprises a hydroxyapatite. Hydroxyapatites are often used in medical devices as they may have one or more of the following properties: stability, biocompatibility, rapid integration with the human body, non-toxicity, non-thrombogenicity, angiogenicity, and is not likely to induce inflammatory reactions. Exemplary hydroxyapatites include those disclosed in U.S. Pat. No. 6,426,114 and U.S. Publication No. 20060134160, the disclosures of which are incorporated herein by reference. In one embodiment, the hydroxyapatite is a porous hydroxyapatite.

### Coatings

“Coated medical device” as used herein includes those devices having one or more coatings, i.e., at least one coating. The at least one coating can comprise one coating covering at least a portion of the device, e.g., all or some of the device. For example, where the device is a stent, the coating can cover the entire stent, or can cover only the portion of the stent that contacts a body lumen. The device may employ more than one coating for different portions of the device, or can employ multiple layers of coatings.

The bilayer composition can be applied onto the medical device by any means known in the art. For example, the medical device can be dipped in the solution, suspension, or emulsion containing the drug and lipid bilayer composition. Alternatively, the bilayer/drug-containing solution, suspension, or emulsion can be sprayed or brushed on the surface of the stent. The coating can then be dried, e.g., by applying a vacuum, to form the dry film on the stent. Other coating methods include rolling, brushing, electrostatic plating, spinning, or inject printing. The compositions can be applied by these methods either as a solid (e.g., film or particles), a suspension, as a solution.

In another embodiment, the solution or emulsion can be formed into particles and applied to the medical device by any technique known in the art, such as, injection, dipping, solvent evaporation from emulsions, and spraying, such as air spraying including atomized spray coating, and spray coating using an ultrasonic nozzle.

In one embodiment, the medical device is prepared by coating at least a portion of the device with at least one lipid bilayer and a bisphosphonate. The coating can comprise combining at least one lipid with the bisphosphonate, such as in solution.

In one embodiment, the combining comprises forming a solution, suspension, or emulsion containing the lipid(s) and agent(s) followed by coating onto the device by any number of methods. In one embodiment, at least one additional pharmaceutically active agent can be combined with the bisphosphonate/lipid solution.

In another embodiment, where the at least one coating comprises an additional pharmaceutically active agent, the at least one pharmaceutically active agent can be combined with the at least one lipid and bisphosphonate to form a dry film comprising both the bisphosphonate and additional agent. In another embodiment, the coating can comprise separately coating the device with the dry film subsequent or prior to coating the device with the additional agent. In one embodiment, the device is first coated with an inner layer, such as those disclosed here (e.g., a hydroxyapatite such as a porous hydroxyapatite) prior to coating with the bisphosphonate and optionally at least one additionally pharmaceutically active agent.

In one embodiment, the at least one additional pharmaceutically acceptable agent is present in the dry lipid film. In another embodiment, the at least one coating comprises a dry film as one layer, and the additional agent deposited in molecular form (e.g., applied as a solution and then dried) and is thus, external to the dry film although may contact the film. In one embodiment, the coating is designed to allow the bisphosphonate and additional agent to be delivered as encapsulated in a liposome, e.g., by including both the bisphosphonate and additional agent in the dry film. In another embodiment, only the bisphosphonate is liposome-encapsulated and the additional agent is released unencapsulated, e.g., by applying the additional agent external to the dry film.

In one embodiment, one or more layers of dry film can be coated onto the device, e.g., a stent. For example, one layer can contain a first pharmaceutically active agent, and a second layer can contain a second pharmaceutically active agent. Additional agents can be contemplated in the first or second layer or in one or more additional layers.

In one embodiment, the bilayer/drug-containing composition is applied to the surface of the medical device. Alternatively, the device can be coated with a first substance that is capable of absorbing the bilayer/drug-containing composition. In another embodiment, the device can be constructed from a material comprising a biocompatible polymer, as disclosed herein.

In one embodiment, the coatings disclosed herein are applied to a device by first coating the substrate, e.g., a ceramic, on the surface of the device, followed by coating the lipid bilayer/agent film on all or a portion of the substrate.

### Pharmaceutically Active Agents

The at least one pharmaceutically acceptable agent can be selected from one or more therapeutically effective agents known in the industry. They can take the form of organic compounds and pharmaceuticals, recombinant DNA and RNA products, collagens and derivatives, proteins and analogs, saccharides and analogs and derivatives thereof.

In one embodiment, the at least one pharmaceutically active agent is selected from anti-inflammatory agents, anti-proliferatives, pro-healing agents, gene therapy agents, extracellular matrix modulators, anti-thrombotic agents/anti-platelet agents, antiangioplastic agents, antisense agents, anticoagulants, antibiotics, bone morphogenetic proteins, integrins (peptides), and disintegrins (peptides and proteins).

Exemplary anti-inflammatory agents include pimecrolimus, adrenocortical steroids (e.g., cortisol, cortisone, fludrocortisone, prednisone, prednisolone, 6α-methylprednisolone, triamcinolone, betamethasone, and dexamethasone), non-steroidal agents (salicylic acid derivatives such as aspirin, para-aminophenol derivatives such as acetaminophen, indole and indene acetic acids (e.g., indomethacin, sulindac, and etodalac), heteroaryl acetic acids (e.g., tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone). Exemplary anti-proliferatives include sirolimus, everolimus, actinomycin D (ActD), taxol, and paclitaxel. Exemplary pro-healing agents include estradiol. Exemplary gene therapy agents include gene delivering vectors e.g., VEGF gene, and c-myc antisense. Exemplary extracellular matrix modulators include batimastat. Exemplary anti-thrombotic agents/anti-platelet agents include sodium heparin, low molecular weight heparin, hirudin, argatroban, forskolin, vapiprost, prostacyclin and prostacyclin analogs, dextran, D-phe-pro-arg-chloromethylketone (e.g., synthetic antithrombin), dipyridamole, glycoprotein IIb/IIIa platelet membrane receptor antagonist, recombinant hirudin, and thrombin inhibitor. Exemplary antiangioplastic agents include thiophosphoramide. Exemplary antisense agents include oligionucleotides and combinations. Exemplary anticoagulants include hirudin, heparin, synthetic heparin salts and other inhibitors of thrombin. Exemplary antibiotics include vancomycin, dactinomycin (e.g., actinomycin D), daunorubicin, doxorubicin, and idarubicin. Exemplary disintegrins include saxatilin peptide. Derivatives and analogs thereof of these examples are also included.

Other exemplary classes of agents include agents that inhibit restenosis, smooth muscle cell inhibitors, immunosuppressive agents, and anti-antigenic agents.

Exemplary drugs include paclitaxel, sirolimus, everolimus, tacrolimus, biolimus, pimecrolimus, midostaurin, bisphosphonates (e.g., zoledronic acid), heparin, gentamycin, and imatinib mesylate (gleevec).

In one embodiment, the at least one pharmaceutically active agent is selected from bisphosphonates. Biologically, bisphosphonates have the potential for modulating inflammatory responses and thus can have anti-inflammatory and anti-arthritic properties. The anti-inflammatory effects of bisphosphonates derive from their effect on macrophages. In one embodiment, the at least one coating comprising a lipid bilayer and bisphosphonate (i.e., one or more bisphosphonates) provide a means for delivering a liposome-encapsulated bisphosphonate (and optionally other therapeutic agents) into macrophages for a treatment regimen.

Bisphosphonates are also known to attach to the mineralized matrix of bone and inhibit bone resorption, e.g., by inhibiting the formation, aggregation, and dissolution of calcium phosphate crystals. Accordingly, they are used in treating pathological conditions involving bone resorption, such as Paget's disease, malignant hypocalcaemia, ostrolytic bone metastasis, and fibrous dysplasia of bone.

Exemplary bisphosphonates include etidronate, clodronate, pamidronate, alendronate, risedronate, tiludronate, ibandronate, zoledronate, incadronate, olpadronate, neridronate, minodronate, YH 529, and EB-1053. As the actual form of the bisphosphonate depends on the pH of the solution, “bisphosphonate” as used herein also include the corresponding acid.

Zoledronic acid (or zoledronate) is a bisphosphonate that belongs to a new class of potent bisphosphonates. Because zoledronic acid is hydrophilic, the liposome provides a hydrophobic matrix to deliver the zoledronic acid in a physiological medium to a target site. Liposome-encapsulated zoledronic acid can be phagocytosed by infiltrating macrophages, and can effectively poison their energy pathway and shut down macrophage activity without substantially harming luminal endothelial cells or affecting endothelial growth.

The concentration of the drug in the lipid film is tailored depending on the specific target cell, disease extent, lumen type, etc. In one embodiment, the concentration of drug in the lipid film can range from 0.001% to 75% by weight relative to the total weight of the solid film, such as a concentration of 0.1% to 50% by weight relative to the total weight of the solid film. In another embodiment, the concentration of drug in the lipid film can range from 0.01% to 40% by weight, such as a concentration ranging from 0.1% to 20% by weight relative to the total weight of the solid film. In another embodiment, the concentration of drug in the lipid film range from 1% to 50%, 2% to 45%, 5% to 40%, or 10% to 35% by weight, relative to the total weight of the solid film. In another embodiment, the drug load can range from 0.1 ng to 5 μg per mm length of a given stent configuration, such as a drug load ranging from 1 ng to 5 μg, or from 0.1 ng to 1 μg, or from 1 ng to 1 μg, or from 0.1 ng to 100 ng or from 0.1 μg to 5 μg, or from 0.1 μg to 1 μg, or from or from 1 μg to 5 μg.

### Devices

Exemplary devices include sutures, staples, anastomosis devices, vertebral disks, bone pins, suture anchors, hemostatic barriers, clamps, screws, plates, clips, vascular implants, urological implants, tissue adhesives and sealants, tissue scaffolds, bone substitutes, intraluminal devices, and vascular supports. For example, the device can be a cardiovascular device, such as venous catheters, venous ports, tunneled venous catheters, chronic infusion lines or ports, including hepatic artery infusion catheters, pacemakers and pace maker leads, and implantable defibrillators. Alternatively, the device can be a neurologic/neurosurgical device such as ventricular peritoneal shunts, ventricular atrial shunts, nerve stimulator devices, dural patches and implants to prevent epidural fibrosis post-laminectomy, devices for continuous subarachnoid infusions, and biodegradable discs eluting i.e. imatinib, implanted after brain tumor removal. The device can be a gastrointestinal device, such as chronic indwelling catheters, feeding tubes, portosystemic shunts, shunts for ascites, peritoneal implants for drug delivery, peritoneal dialysis catheters, and suspensions or dry implants to prevent surgical adhesions. In another example, the device can be a genitourinary device, such as uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy, central venous catheters.

Other exemplary devices include prosthetic heart valves, vascular grafts ophthalmologic implants (e.g., multino (molteno) implants and other implants for neovascular glaucoma, drug eluting contact lenses for pterygiums, splints for failed dacrocystalrhinostomy, drug eluting contact lenses for corneal neovascularity, implants for diabetic retinopathy, drug eluting contact lenses for high risk corneal transplants), otolaryngology devices (e.g., ossicular implants, Eustachian tube splints or stents for glue ear or chronic otitis as an alternative to transtempanic drains), plastic surgery implants (e.g., breast implants or chin implants), and catheter cuffs and orthopedic implants (e.g., cemented orthopedic prostheses).

Another exemplary device according to the invention is a stent, such as a stent comprising a generally tubular structure. A stent is commonly used as a tubular structure disposed inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

An exemplary stent is a stent for treating narrowing or obstruction of a body passageway in a human or animal in need thereof. “Body passageway” as used herein refers to any of number of passageways, tubes, pipes, tracts, canals, sinuses or conduits which have an inner lumen and allow the flow of materials within the body. Representative examples of body passageways include arteries and veins, lacrimal ducts, the trachea, bronchi, bronchiole, nasal passages (including the sinuses) and other airways, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina and other passageways of the female reproductive tract, the vasdeferens and other passageways of the male reproductive tract, and the ventricular system (cerebrospinal fluid) of the brain and the spinal cord. Exemplary devices of the invention are for these above-mentioned body passageways, such as stents, e.g., vascular stents. There is a multiplicity of different vascular stents known in the art that may be utilized following percutaneous transluminal coronary angioplasty.

In another embodiment, the device is a stent and coating comprises a lipid bilayer film coating a substrate, and the thickness of the substrate is selected to provide a sufficiently flexible coating that stays adhered to the stent even during mounting and expansion of the stent. A typical mounting process involves crimping the mesh-like stent onto a balloon of a catheter, thereby reducing its diameter by 75%, 65%, or even 50% of its original diameter. When the balloon mounted stent is expanded to place the stent adjacent a wall of a body lumen, e.g., an arterial lumen wall, the stent, in the case of stainless steel, can expand to up to twice or even three times its crimped diameter. For example, a stent having an original diameter of 1.7 mm can be crimped to a reduced diameter of 1.0 mm. The stent can then be expanded from the crimped diameter of 1.0 mm to 3.0 mm. Accordingly, in one embodiment, the substrate has a thickness of no more than 2 μm, such as a thickness of no more than 1 μm.

Alternatively, any number of medical devices or stents may be utilized in accordance with the present invention and the invention is not limited to the specific stents that are described in exemplary embodiments of the present invention. In addition, as stated above, other medical devices may be utilized, such as e.g., orthopedic implants. The stent or medical device can be made of various materials including stainless steel, CoCr, titanium, titanium alloys, NiTi, and polymers typically used for implantable medical devices. Exemplary polymers include polyurethanes, polyacrylate esters, polyacrylic acid, polyvinyl acetate, silicones, styrene-isobutylene-styrene block copolymers such as styrene-isobutylene-styrene tert-block copolymers (SIBS); polyvinylpyrrolidone including cross-linked polyvinyl pyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polycarbonates, siloxane polymers; cellulosic polymers such as cellulose acetate; and mixtures and copolymers of any of the foregoing. In one embodiment, the nonbiodegradable polymer is selected from poly(n-butyl methacrylate)/poly(ethene vinyl acetate), polyacrylate, poly(lactide-co-E-caprolactone), PTFE, paralyene C, polyethylene-co-vinyl acetate, poly n-butylmethacrylate, poly(styrene-b-isobutylene-b-styrene) (a tri-block copolymer of styrene and isobutylene subunits built on 1,3-di(2-methoxy-2-propyl)-5-tert-butylbenzene, Transelute™).

In one embodiment the medical device is a porous structure, e.g. a porous orthopedic prostheses. The lipid film can be applied within the porosity of the device using techniques known to the art such as, but not limited to, dipping, spraying, or brushing. In other embodiments the surface of the device can be porous or made porous using techniques known to the art, such as electroplating, and the lipid film can be further applied in the porosity of the surface using techniques mentioned above. In other embodiments a porous inner coating can be applied on the surface of the medical device such as a porous hydroxyapatite coating. The lipid film can be further applied to the porous surface.

### Methods of Treating Diseases

One embodiment provides a method of treating at least one disease or condition comprising:

implanting in a subject in need thereof a medical device comprising a coating covering at least a portion of the device, the coating comprising at least one lipid bilayer and a therapeutically effective amount of at least one pharmaceutically active agent, and

releasing from the device the at least one pharmaceutically active agent encapsulated in a liposome comprising lipids from the lipid bilayer.

In one embodiment, the at least one disease or condition is a proliferative disorder (e.g., a tumor), an inflammatory disease, or an autoimmune disease.

In one embodiment, the device is useful for treating diseases or conditions associated with the narrowing or obstruction of a body passageway in a subject in need thereof. In one embodiment, the disease or condition is associated with restenosis. In one embodiment, the at least one disease or condition is neointima and neointimal hyperplasia. In another embodiment, the at least one disease or condition is selected from thrombosis, embolism, and platelet accumulation. In yet another embodiment, the disease or disorder is the proliferation of smooth muscle cells.

In one embodiment, the bilayer composition can be chosen to release the agent over a desired period of time, e.g., days or less, or weeks to months. Accordingly, one embodiment provides a bilayer film that releases the at least one pharmaceutically active agent over a period of 7 days or less, such as a period of 3 days or less. In another embodiment, the bilayer film releases the at least one pharmaceutically active agent over a period from at least 7 days, or at least 10 days and even up to a period of 1 year, e.g., from 1 week to 1 year, such as a period ranging from 2 weeks to 6 months. In another embodiment, the bilayer film releases the at least one pharmaceutically active agent over a period ranging from 7 days to 6 months, from 7 days to 3 months, from 7 days to 2 months, from 7 days to 1 month, from 10 days to 1 year, from 10 days to 6 months, from 10 days to 2 months, or from 10 days to 1 month.

In one embodiment, the method comprises inserting the device into the passageway, the device comprising a generally tubular structure, the surface of the structure being coated with a composition disclosed herein, such that the passageway is expanded. In the method, the body passageway may be selected from arteries, veins, lacrimal ducts, trachea, bronchi, bronchiole, nasal passages, sinuses, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina, the vasdeferens, and the ventricular system.

In one embodiment, the implantable devices disclosed herein are implanted in a subject in need thereof to achieve a therapeutic effect, e.g., therapeutic treatment and/or prophylactic/preventative measures. Those in need of treatment may include individuals already having a particular medical disease as well as those at risk for the disease (e.g., those who are likely to ultimately acquire the disorder). A therapeutic method can also result in the prevention or amelioration of symptoms, or an otherwise desired biological outcome, and may be evaluated by improved clinical signs, delayed onset of disease, reduced/elevated levels of lymphocytes and/or antibodies.

In one embodiment, the method comprises inserting an implantable medical device in the form of vascular stent into a blood vessel, the stent having a generally tubular structure, the surface of the structure being coated with a composition as described above, such that the vascular obstruction is eliminated. For example, stents may be placed in a wide array of blood vessels, both arteries and veins, to prevent recurrent stenosis (restenosis) at, e.g., a site of (failed) angioplasties, to treat narrowings that would likely fail if treated with angioplasty, and to treat post surgical narrowings (e.g., dialysis graft stenosis).

### EXAMPLES

### Example 1

This Example describes a method for producing a dry phospholipid film. Liposome formation from the film in aqueous solution is observed optically. L-α-Phosphatidylcholine (PC, from soybean) and cholesterol were dissolved in dichloromethane. Paclitaxel (PTX), as a model hydrophobic drug, was added to this solution to produce Formulation B. The weight percent of cholesterol in Formulation B is 10% of the total lipid. The precise amounts of various components are listed in Table 1.

TABLE 1
Composition of Formulation B

| Ingredients | Formulation B |
|---|---|
| PC (g) | 0.18 |
| Cholesterol (g) | 0.02 |
| Paclitaxel (g) | 0.07 |
| DCM (mL) | 10 |
| Total of solid | 0.27 |
| phase (g) | |

Formulation B was sprayed on a stainless tube and the tube was placed in a vacuum oven (Napco model 5831, Thermo Electron Corporation) for 12 hours at 30 in. Hg to remove solvent at room temperature. The tube was placed in 1 ml of phosphate buffer solution (PBS). Optical micrographs were obtained from the coating at different time intervals using an inverted optical microscope (Vistavision, VWR). Optical micrographs of a stainless tube coated with formulation B and immersed in PBS are shown immediately upon immersion (FIG. 2A), 30 minutes after immersion (FIG. 2B), and 60 minutes after immersion (FIG. 2C) at a magnification of approximately 40×. Liposome formation can be readily seen as translucent globules, as indicated by the arrows of FIGS. 2B and 2C, and are initially nearly absent in the lipid film of FIG. 2A.

### Example 2

This Example describes the preparation of a lipid film comprising a hydrophobic drug and the ability to tailor the amount of liposome formation by changing the amounts of lipid in the dry film. Two different formulations (Formulations A and B) were prepared comprising a mixture of lipids L-α-phosphatidylcholine (PC, from soybean) and cholesterol dissolved in dichloromethane. The weight percent of cholesterol in Formulation A was 30% of the total lipid and in Formulation B is 10%, and the precise amounts are listed in Table 2. Paclitaxel (PTX), as a model hydrophobic drug, was added to this solution. One hundred μL of the above solution added to a round bottom tube and was dried under vacuum (30 inches Hg) for 12 hours in order to remove solvents, simulating the formation of a lipid film on the surface of a substrate.

TABLE 2
Composition of Formulations A and B

| Ingredients | Formulation A | Formulation B |
|---|---|---|
| PC (g) | 0.14 | 0.18 |
| Cholesterol (g) | 0.06 | 0.02 |
| Paclitaxel (g) | 0.07 | 0.07 |
| DCM (mL) | 5 | 5 |
| Total of solid | 0.27 | 0.27 |
| phase (g) | | |

To the resulting lipid film, 10 mL of phosphate buffer solution (PBS) was added and the glass vial was placed on a rotating apparatus at a rotation speed of 20 rpm operating in a water bath kept at 37.2° C. The contents of the glass tube (eluted liquid) was emptied into a 15 ml eppendorf tube over several intervals and the glass tube was filled with fresh PBS and placed back in the rotating apparatus. Twenty μL of the eluted liquid was examined optically using an inverted microscope as described in Example 1. The rest of the eluted liquid was centrifuged for 30 minutes at 4000 rpm to separate the lipids from the liquid. The supernatant was removed and was analyzed with HPLC for PTX. The lipid content was dissolved in 2 ml ethanol and was also analyzed with HPLC.

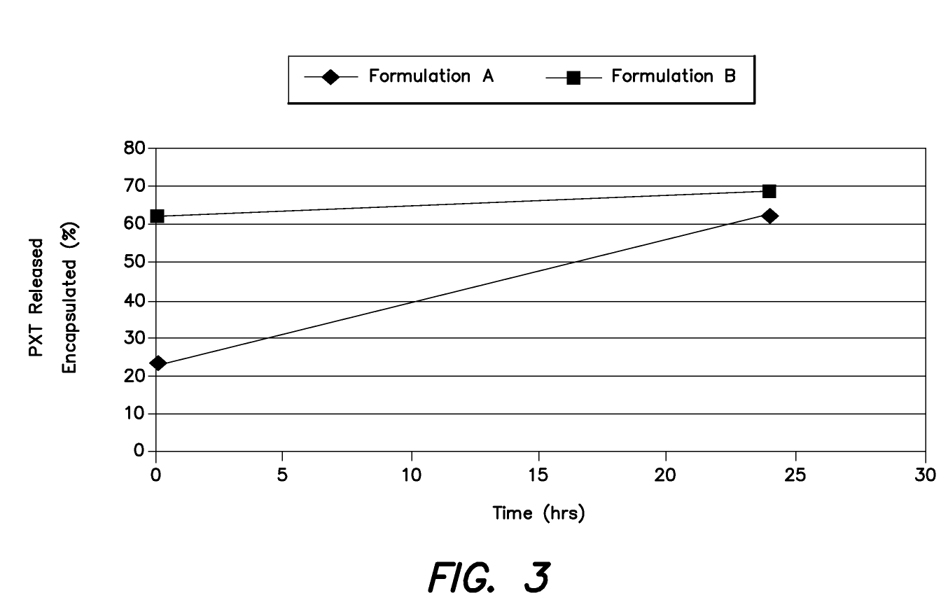

The results of the HPLC analysis are presented in FIG. 3, which shows a graph of the amount of liposome encapsulated paclitaxel released over time for Formulation A (♦) and Formulation B (•). At the 1 hr time period, HPLC analysis of the supernatant and the lipid content of the eluted liquid showed significantly higher encapsulation percentage in case of Formulation B in comparison with Formulation A. The total amount of drug released was similar for the A and B compositions (70 μg and 76 μg, respectively).

At 24 hr, the percentage of encapsulated drug increased significantly for Formulation A and increased slightly for Formulation B. The total amount of released drug remained similar for the formulations A and B (16 μg and 17 μg, respectively).

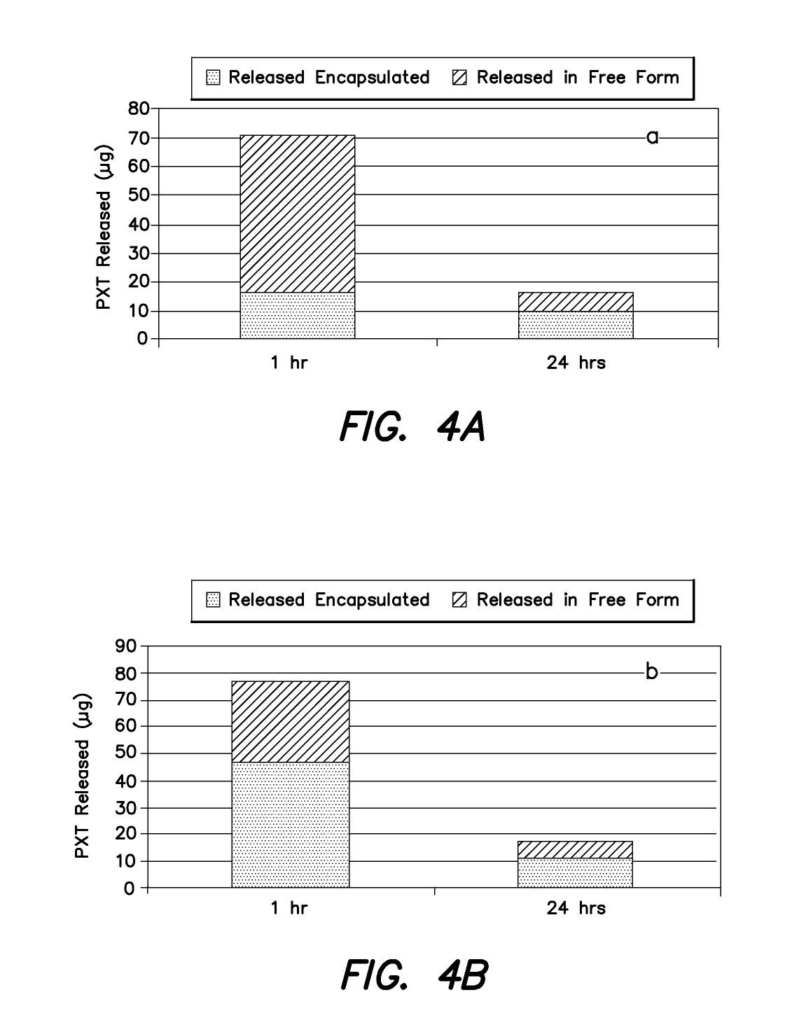

FIGS. 4A (Formulation A) and 4B (Formulation B) show bar charts comparing the amount of released drug as encapsulated versus unencapsulated at 1 h and 24 h. It was observed that the elution profile of the lipid film, irrespective of the composition of the lipid film, had an initial burst period which further slows down with time. For Formulation A, the percentage of drug released in the free form decreased with time suggesting that the initial drug release in the first hour of elution was mainly controlled by a diffusion mechanism.

It was observed that the composition of the lipid film affects the amount of the drug that is released in encapsulated form. This Example demonstrates that the addition of cholesterol to the phospholipid can significantly affect the percentage of a drug released in an encapsulated form. Formulation B, which contained 10 weight percent cholesterol, provided a higher percentage of encapsulated drug compared to Formulation A, which contained 30 weight percent cholesterol.

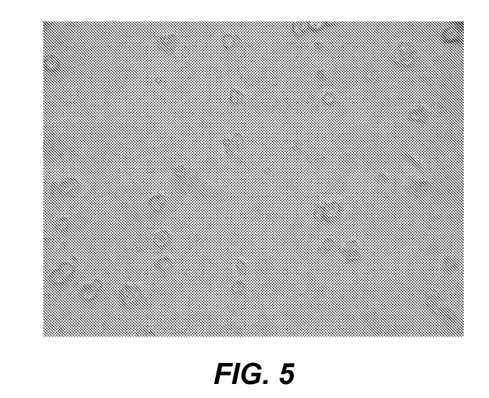

Formulation B, produced a significant amount of bilayer structures upon hydration and a significant amount of drug (>60%) was released encapsulated in the bilayer structures. FIG. 5 is an optical micrograph of Formulation B after 1 h, showing the liposomes as translucent globules.

### Example 3

This Example demonstrates the formation of a lipid film comprising a hydrophilic drug via an emulsion method.

Lecithin (0.14 g) and cholesterol (0.06 g) are dissolved in 9 mL of dichloromethane (solution 1). Imatinib mesylate (0.0641 g) is dissolved in 200 μL of distilled water (solution 2). Solution 2 is added drop-wise into solution 1 to form an (emulsion 1). Ethanol (2 mL) is added to emulsion 1 to obtain a clear emulsion. The emulsion is then applied on the surface of the medical device.

### Example 4

This Example demonstrates the preparation of a solution for forming a lipid film containing zoledronic acid (ZA).

First, 70 mg of ZA is dissolved in 500 μL of 1 N NaOH, resulting in a pH of about 8. A lipid solution of L-α-Lecithin (0.24 g) and cholesterol (0.06 g) are dissolved in 9 mL methylene chloride. 100 μL of the ZA solution is added to the lipid solution. The emulsion is sonicated at 40±2° C. until a clear solution is obtained. This solution does not remain clear in room temperature and the two phases separate in approx 5 min.

To stabilize the emulsion at room temperature, the above clear solution is heated at 60±2° C. until the clarity of the solution diminishes. It is then removed from the heat and is stirred at room temperature until the cloudiness disappears and a clear, transparent solution is obtained. This solution remains stable at room temperature and is suitable for further coating processes.

To characterize the drug content, 100 μL of the lipid formulation is dried in a vacuum chamber operated at −25 mmHg for 1 hour. The dried film is weighed and then dissolved in 1 ml of 2% (wt/vol) Triton X solution by sonication at 40±2° C. for 5 min. A sample of the solution is tested for drug content using HPLC. The percent of ZA in the formulation is calculated using the following equation:

% drug=amount of drug measured by HPLC/weight of the coating×100

### Example 5

This Example describes the preparation of hydroxyapatite (HAp)-coated stents further coated with a lipid formulation.

Metallic stents were coated with porous hydroxyapatite (HAp). Any porous hydroxyapatite is suitable for this experiment, including those porous hydroxyapatites described in U.S. Publication No. US20060134160, the disclosure of which is incorporated herein by reference. HAp coated stents are dipped in the lipid formulation of Example 4 solution) for 1 min 30 seconds. They are further removed from the solution and are spun with a rotary device at a speed of 5000 rpm to remove extra solution from the surface of the stent. The stents are further placed inside a vacuum chamber at −25 mmHg for 12 hours to remove extra solvents.

To determine the amount of the drug in the coatings, HAp coated stents were weighed before and after coating. The weight of the coating were determined to be 100-120 μg. The coatings were further dissolved using 1 ml of 2% Triton X solution and was analyzed using HPLC machine for ZA content. The coatings contained 2 μg of ZA.

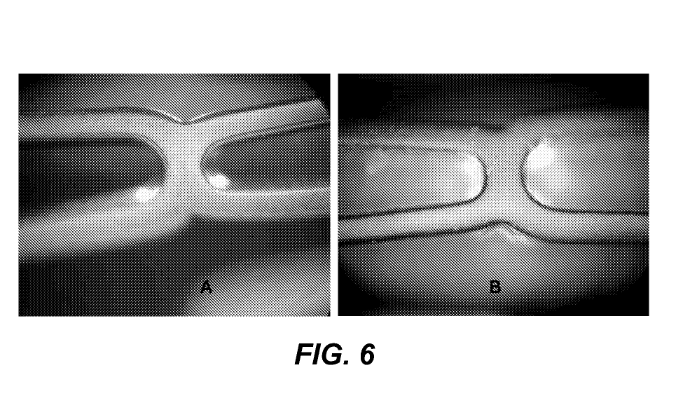

FIG. 6 shows the optical pictures of porous HAp coated stents before (A) and after (B) the application of the lipid formulation of Example 4. Even after the stent is coated, the rough surface of the porous HAp coating is still visible.

### Example 6

This Example demonstrates the biological activity of the lipid formulation against Acute Monocytic Leukemia Cell Line—THP-1, purchased from ATCC (Catalog No. TIB-202™) and cultured at 37° C. in complete RPMI-1640 medium (ATCC).

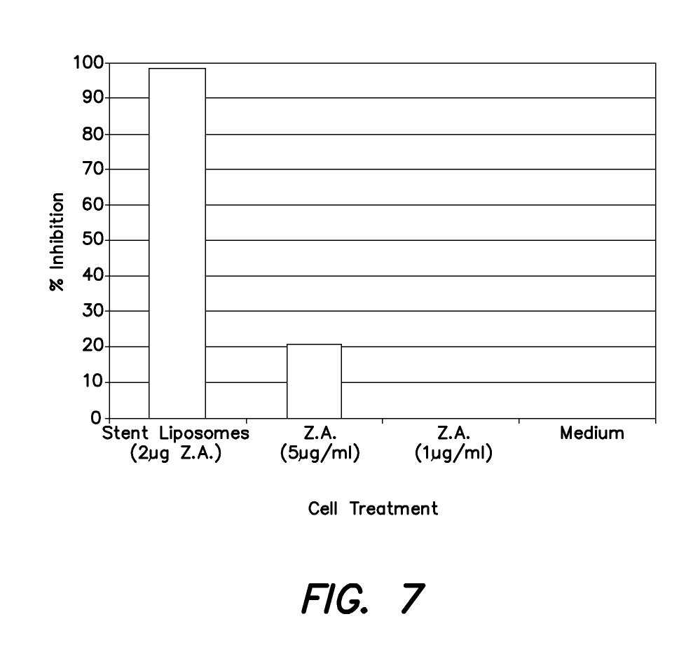

To monitor cell growth in the presence of liposomes containing ZA, 5×10⁵ THP-1 cells are plated into 24—well cluster plates (Corning Inc.) in complete RPMI-1640 medium at 1 mL volume. About 3-4 hours later, the lipid coated stents of Example 5 are added into the THP-1 cell culture (5×10⁵ cells/mL). The cells in the presence of medium alone or treated with empty liposomes (no drug) served as a control. ZA in molecular form was added for comparison at a concentration of 5 and 1 μg/mL. After 4 days of culture, the cells in each culture were harvested and counted using a hemocytometer; viability was evaluated by trypan blue exclusion method. As presented in FIG. 7, which shows a graph of % cell growth inhibition (y-axis) versus cell treatment (x-axis), the coated stent containing ZA in a lipid formulation is much more potent in the inhibition of THP-1 cells as compared to ZA in molecular form; the 2 μg dose of ZA delivered from the lipid coated stents results in 98% of cell growth inhibition, while even a higher dose (5 μg/mL) of ZA in molecular form results in only 20% of cell growth inhibition while 1 μg/mL dose of ZA results in only 0.6% of inhibition.

### Example 7

This Example compares the activity of stents coated with the lipid formulation (containing ZA) of Example 4 versus stents coated with molecular ZA.

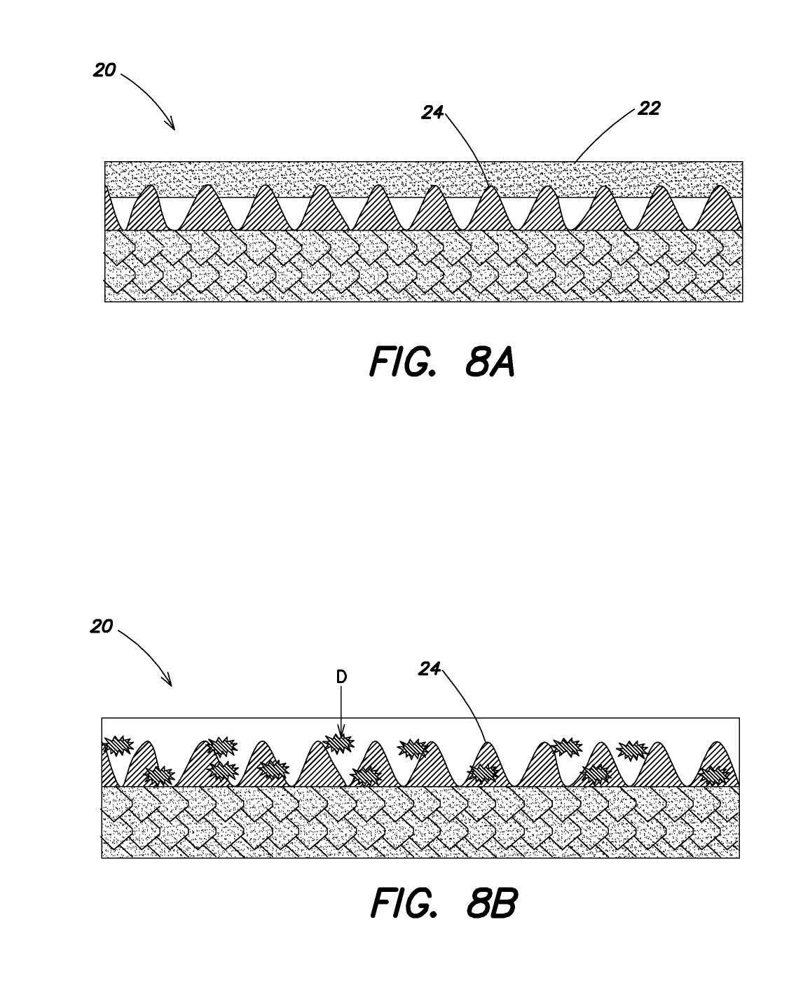

Hydroxyapatite coated stents were coated with liposomal ZA as per Examples 4 and 5. This lipid coating had a weight of 150 μg and contained approximately 3 μg of ZA. To compare the effect of liposomal ZA compared to that of molecular ZA, hydroxyapatite-coated stents were dip coated into a 2.5% ZA in 0.1 N NaOH (pH=7.8) solution. The samples are weighed before and after the dip coating in the ZA solution and the amount of the drug deposited on the HAp coating was calculated to be 13 μg. FIG. 8A schematically shows the configuration of a medical device 20, such as a stent, having an HAp coating 24 further coated with the lipid formulation 22 containing ZA. FIG. 8B schematically shows medical device 20 having an HAp coating 24 molecular ZA (“D”) only.

The two sets of samples were placed in cell cultures containing THP-1 macrophage type cells. HAp coated stents impregnated with molecular ZA (configuration B) are placed in 2 ml PBS dissolution media and rotated at a speed of 20 rpm at 37.2° C. Samples are taken at various time intervals and analyzed using HPLC.

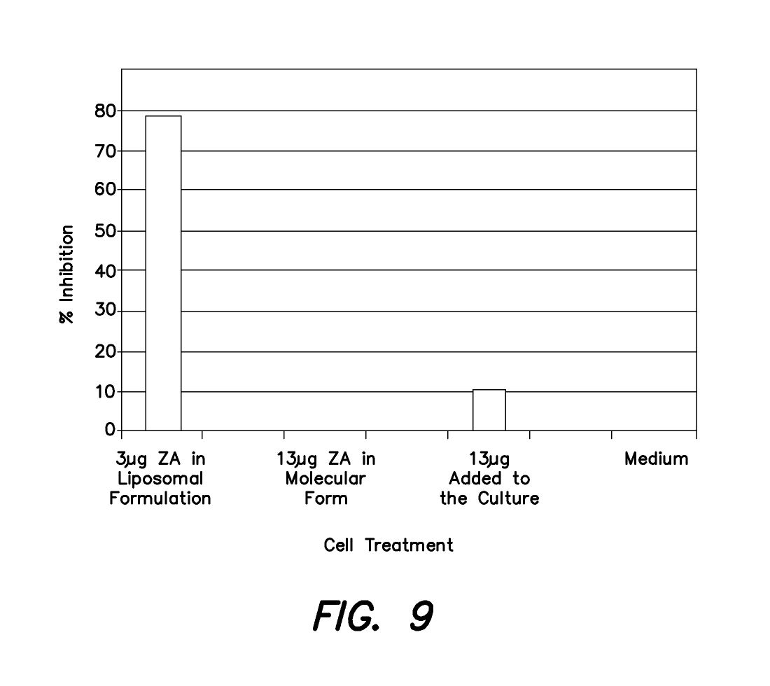

FIG. 9 is a graph showing the inhibitory effect of stents coated with the lipid formulation on porous HAp versus a porous HAp coated stent impregnated with molecular ZA, a sample containing molecular ZA added directly to the culture of THP-1 cells, and a sample containing medium only. For the stent coated with molecular ZA, no ZA was eluted for the period of 5 days of elution test. It is known that ZA has a strong affinity to HAp, which can explain the observation that porous HAp coated stents impregnated with molecular ZA showed no biological activity in the cell culture experiment.

### Example 8

The lipid formulation containing ZA can also be applied on the surface of porous HAp coating by spraying. In this Example, HAp coated stents are loaded into a spray coating machine, the solution is pumped into an ultrasonic nozzle and is sprayed on the stent while the stent is rotated and is moved back and forth underneath the nozzle to achieve coating uniformity.

### Example 9

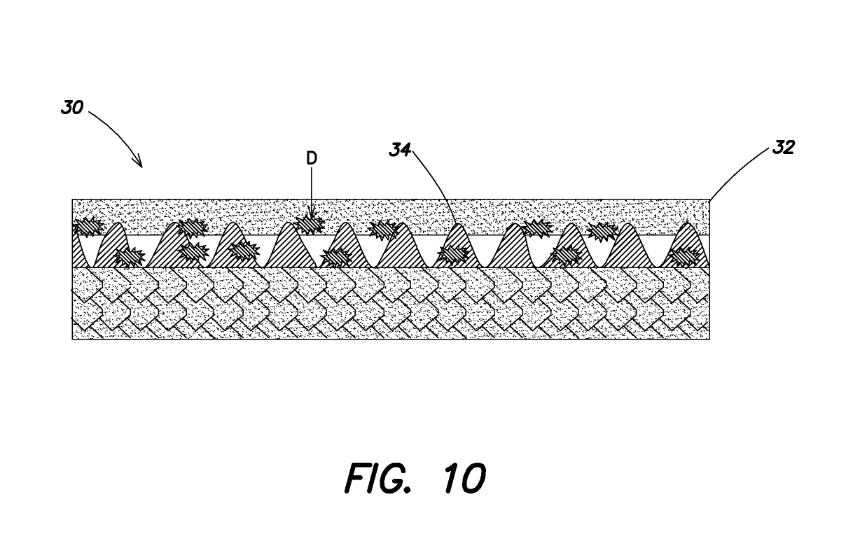

Porous hydroxyapatite coated stents can be impregnated with a variety of drugs or formulation. This Example shows the application of a lipid formulation containing ZA along with another therapeutic agent for additional anti-inflammatory and/or anti-proliferative properties to the coating. In this Example porous HAp coated stents are impregnated with midostaurin. A layer of the lipid formulation of Example 4 is further sprayed on the surface using a spray coating process as described in Example 8. FIG. 10 schematically shows the above configuration where a stent 30 coated with porous HAp 34 is first impregnated with midostaurin (PKC-412, “D”), in which a lipid formulation of ZA 32 is further sprayed on this combination.

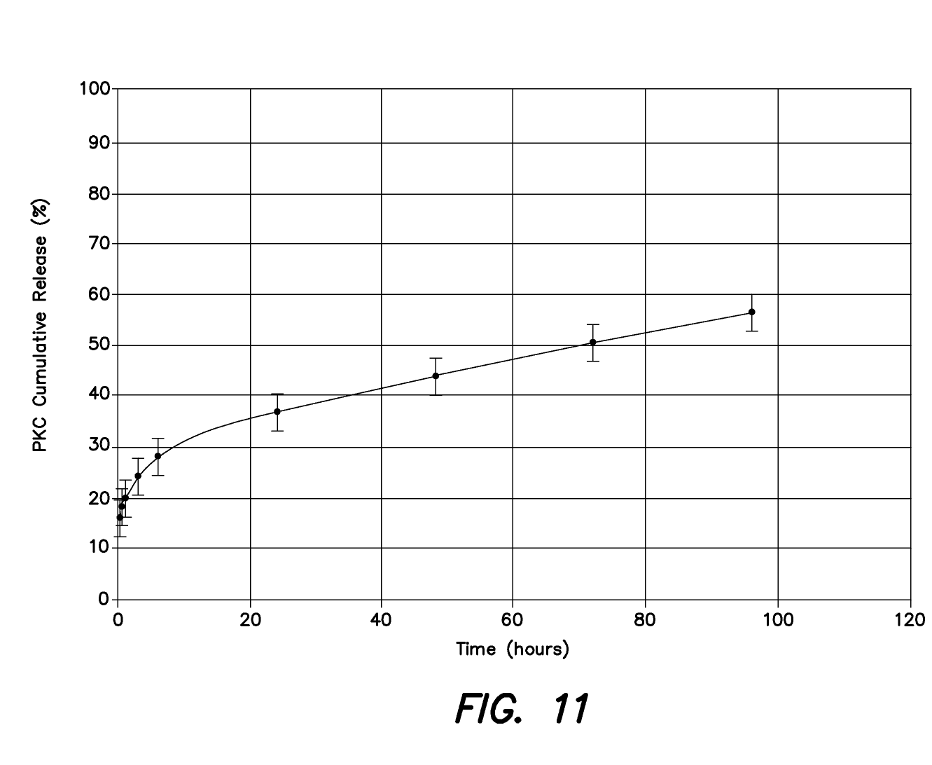

FIG. 11 shows the release profile of midostaurin as a graph of % cumulative release (y-axis) versus time (x-axis) from the combination coating of free midostaurin and liposomal ZA in 10 ml of dissolution media containing PBS plus 0.02% SLS rotating at the speed of 20 rpm at 37.2° C. Samples from dissolution media were analyzed using HPLC at various time intervals. As can be seen from FIG. 11, midostaurin can be released from the stent over an extended period of time.

### Example 10

It is possible to include another hydrophobic drug in the liposomal formulation of ZA. For example it is possible to include midostaurin directly in the liposomal formulation.

L-α-Lecithin (0.24 g), cholesterol (0.06 g), and midostaurin (0.04 g) are dissolved in 9 ml methylene chloride. 100 μL of the ZA solution is added to the lipid solution. The emulsion is sonicated at 40±2° C. until a clear solution is obtained. The solution is further treated thermally as described in Example 4 to stabilize it at room temperature.

This ZA-midostaurin lipid formulation can be applied on bare metal or hydroxyapatite coated stents by dip or spray coating as described herein.

### Example 11

This Example describes a “sandwich” type coating configuration. A lipid coating is provided on a stent by first spray coating the stent with a one or more layers of lipid composition (0.24 g l-α-lecithin and 0.06 g cholesterol in 9 ml DCM). One or more layers of ZA solution (2% ZA solution in 0.1 N NaOH) is then sprayed on this coating followed by spraying one or more layers of the lipid composition again.

### Example 12

This Example describes a solution for forming a film comprising a lipid bilayer.

The solution was prepared from the components listed in Table 3 below (DSPC=distearoyl phosphatidylcholine; DSPG=distearoyl phosphatidylglycerol).

TABLE 3

| Lipid | Weight (mg) |
|---|---|
| DSPC | 16.00 |
| DSPG | 5.41 |
| Cholesterol | 5.00 |

PKC-412 (midostaurin, 0.59 mg) was dissolved in 2 ml of methanol and added to the above composition to form a clear solution.

### Example 13

This Example describes the preparation of a hydroxyapatite-coated stent further coated with a lipid bilayer film.

The hydroxyapatite-coated stent was prepared as described in U.S. Provisional Application No. 60/978,988, filed Oct. 10, 2007, the disclosure of which is incorporated herein by reference. The Examples below can also be performed with other calcium phosphate or hydroxyapatite-coated stents, such as those devices described in U.S. Patent Publication No. 2006/0134160, or in Tsui M., 2007, “Calcium phosphate coatings on coronary stents by electrophoretic deposition,” M.A.Sc. Thesis, Department of Materials Engineering, University of British Columbia, Vancouver, BC., the disclosures of which are incorporated herein by reference.

A hydroxyapatite coated stent was weighed and sprayed with the above vehicle. The coated stent was placed under vacuum (30 mmHg) for 12 hours. The coated stent was then weighed and the weight of the coating was calculated.

### Example 14

This Example describes the monitoring of coating weight loss over time.

Coated stents prepared according to Examples 12 and 13 were placed in water (10 mL) and were shaken on a bench-top shaker. At various time intervals the stents were recovered from the water, dried at 40° C. and their weights were recorded. The water was examined for PKC-412 content and was replaced with fresh water at each time point.

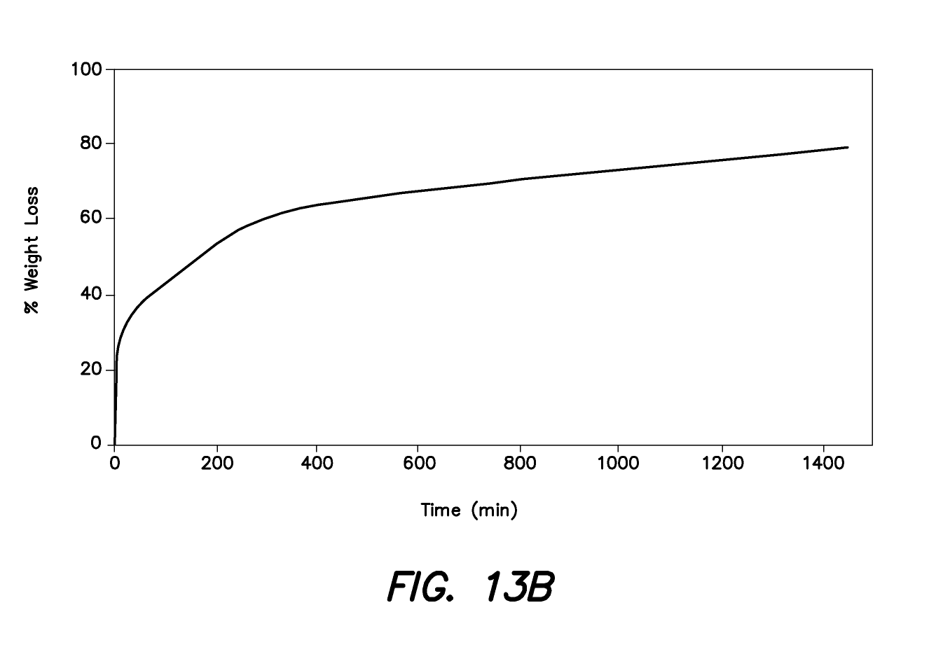

FIG. 13A plots the amount of lipid and drug released (%, y-axis) from the stent over time (min., x-axis). FIG. 13B is a plot showing that the film of this Example can release liposome-encapsulated PKC-412 over a time period of 24 hours. This time period is useful since inflammation processes generally occur within the first few days following implantation of a medical device, such as a stent. The rate of liposome formation can be tailored depending on the lipid type and relative ratios of the components of the lipid bilayer film.

### Example 15

This Example describes the amount of PKC-412 released as a drug-encapsulated-liposome versus free drug.

Coated stents prepared according to Examples 12 and 13 were placed in water (10 mL) and shaken using a bench-top shaker. At various time intervals the stents were recovered from the water. The water was filtered using a 0.1 μm filter and the supernatant was examined for PKC-412 using HPLC. Liposomes collected on the filter were dissolved in methanol and were also examined for PKC-412 using the same HPLC method.

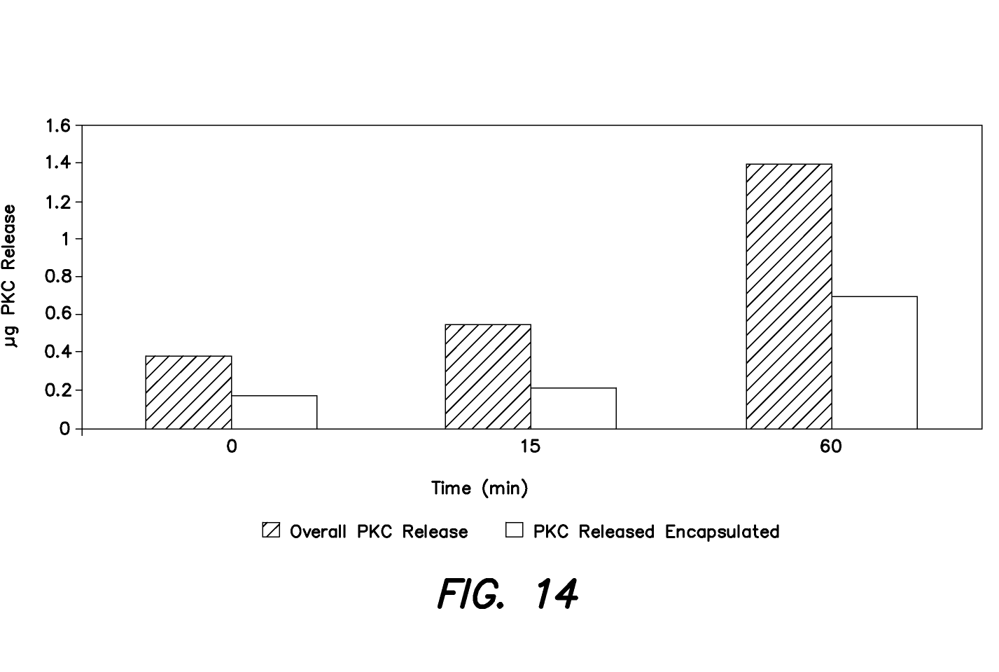

The overall drug release was determined from the amount of free drug recovered from the supernatant and the amount of drug recovered from liposomes. The amount of drug recovered from the filter was assumed to be equal to the amount of encapsulated drug. FIG. 14 is a plot showing the total amount of drug released and the amount of encapsulated PKC-412 released (μg, y-axis) at 0, 15 and 60 minute intervals (x-axis). It can be seen that significant amounts of drug were released in free form and encapsulated in liposomes. This result can serve as a proof of concept that a drug present in a bilayer can be released as both free drug and liposome-encapsulated drug and may show dual functionality in the treatment of both inflammation (primarily by liposomes) and excessive smooth muscle proliferation (primarily by free drug).

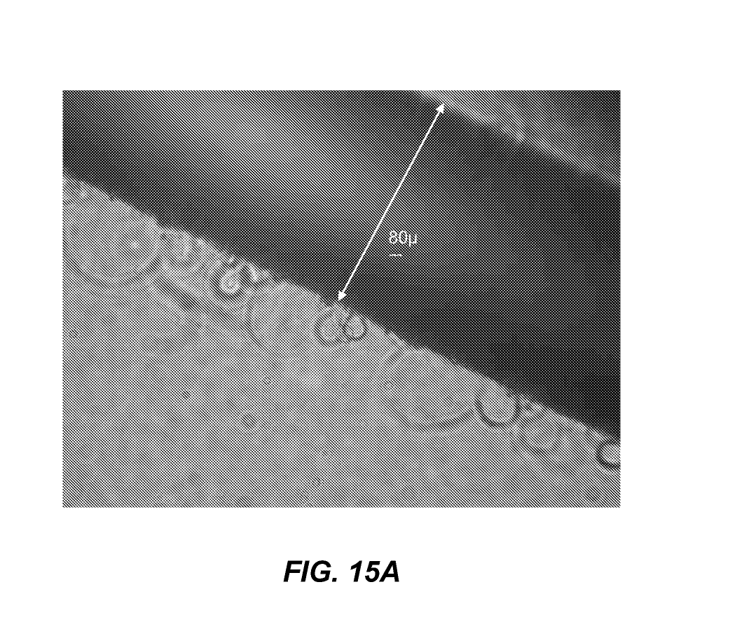

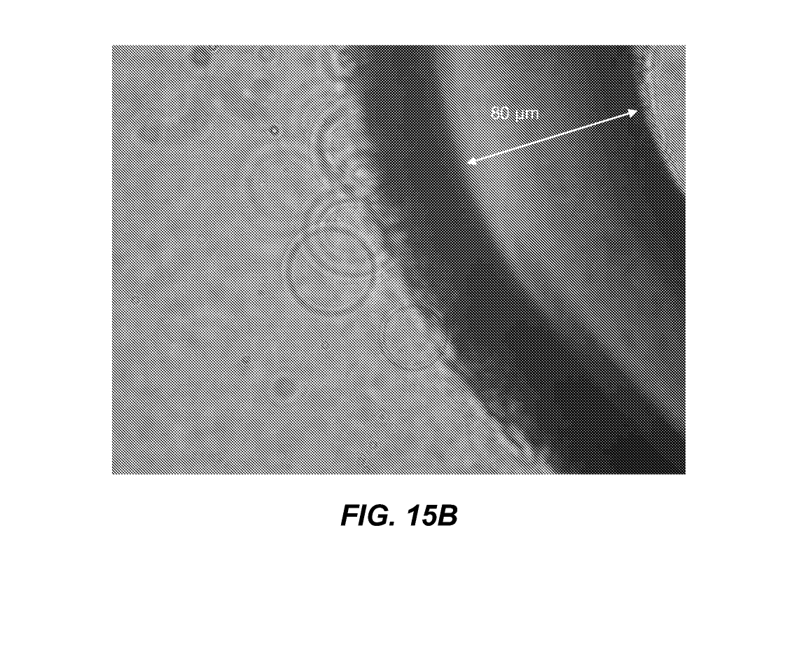

Stents coated with a lipid film as prepared in Examples 12 and 13 were placed in water and observed with an optical microscope. The optical micrographs of FIGS. 15A and 15B (the blackened region is a light shadow) show formed liposomes of various sizes released from the lipid-bilayer coated stents. As the width of the stent strut is approximately 80 μm, it can be seen that a significant population of the liposomes have a diameter greater than 1 μm or 2 μm.

### Example 16

This Example demonstrates the inhibitory effect of zoledronic acid released from a lipid bilayer stent coating after being immersed in an in vitro culture of THP-1 cells.

Hydroxyapatite coated stents were further coated with the lipid film in a manner similar to that described in Examples 12 and 13, except that the film contained 2 μg of zoledronic acid (ZA). The stents were added to THP-1 (Human Monocytic Leukemia) cell culture (5×10⁵ cells/mL) and compared to ZA alone added at concentration of 5 and 1 μg/mL. After 4 days, the cells in each culture were harvested and counted.

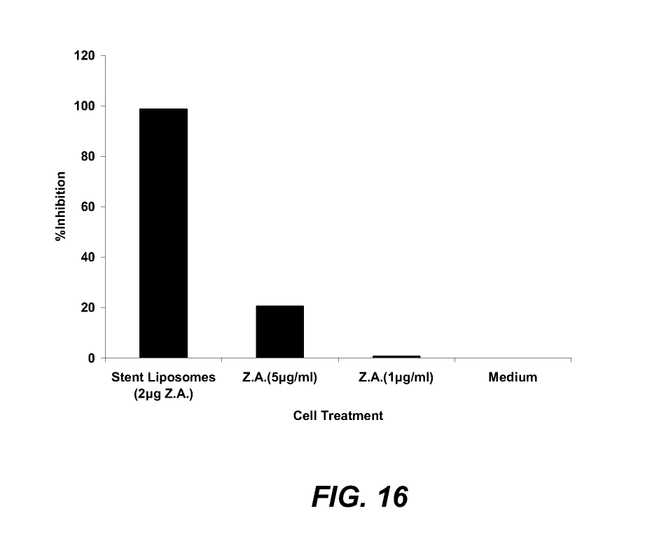

FIG. 16 is a plot of % inhibition of growth of THP-1 for the various ZA samples. ZA released from the lipid bilayer film shows a greater potency in inhibiting THP-1 cells as compared to ZA in molecular form. The 2 μg/mL dose of ZA present in a lipid bilayer film coating the stents resulted in 98% cell growth inhibition, while a higher dose (5 μg/mL) of free ZA produced only 20% of cell growth inhibition and a 1 μg/mL dose of free ZA resulted in only 0.6% inhibition. This experiment demonstrates that liposome-encapsulated ZA has a greater potency toward THP-1 cell growth inhibition compared to free ZA, as demonstrated by the higher potency at a lower dose (2 μg).

ZA is known to be useful in the treatment of plaque vulnerability and restenosis. This Example demonstrates that in addition to the known potencies of ZA, the anti-inflammatory properties of ZA can significantly improve when delivered from a lipid bilayer film. Because ZA is a hydrophilic drug, the use of a bilayer film prevents its premature washout when implanted in the body.

### Example 17

This Example demonstrates the inhibitory effect of midostaurin in liposomal form on in vitro growth of human coronary artery smooth muscle cells (HCASMC).

HCASMC were plated at 5×10⁴ cells/well into 12-well cluster plates (Corning) and cultured overnight in 1 mL of HCASMC growth medium. After 24 hours incubation, 1 mL of medium in each well was replaced with 3 mL of fresh HCASMC growth medium. Stents with an HAp layer coated with a lipid film containing about 2.5 μg of midostaurin were prepared in a manner similar to that of Examples 12 and 13. The stents were inserted into these cell cultures. The process of liposome formation upon contact of stents with growth medium was observed under an inverted light microscope. Wells with medium alone served as a background control.

After one week of incubation, the cells in cultures were harvested by trypsinization. The number of viable cells in each well was counted using a hemocytometer and trypan blue. New cell cultures were started and the stents were transferred into these new cultures for a second week evaluation.

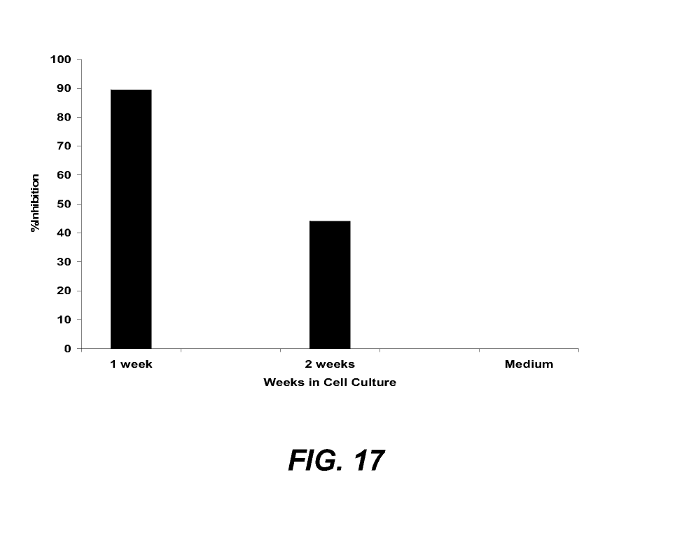

FIG. 17 is a plot of % inhibition of HCASMC growth (y-axis) for one and two week samples and indicates that midostaurin released from the coated stents exposed to a culture medium produced about 90% and 40% of cell growth inhibition after one and two weeks of cell culture, respectively.

### Example 18

This Example demonstrates the growth inhibition of THP-1 cells in the presence of various concentrations of midostaurin in molecular or liposomal form.

Midostaurin was added to in vitro cultures of THP-1 cells (1×10⁶ cells/mL) in concentrations of 0.1, 1, and 4.5 μg/mL. The cultures were maintained in 24-well cluster plate (Corning). Stents coated with lipid film containing 2 μg of midostaurin on HAp layer, as prepared in Examples 12 and 13, were also inserted into cell cultures. Wells with medium alone served as a background control. The process of liposome formation upon contact of stents with growth medium was observed microscopically. After 4 days of incubation, the cell cultures were harvested and the number of viable cells in each well was counted using a hemocytometer and trypan blue.

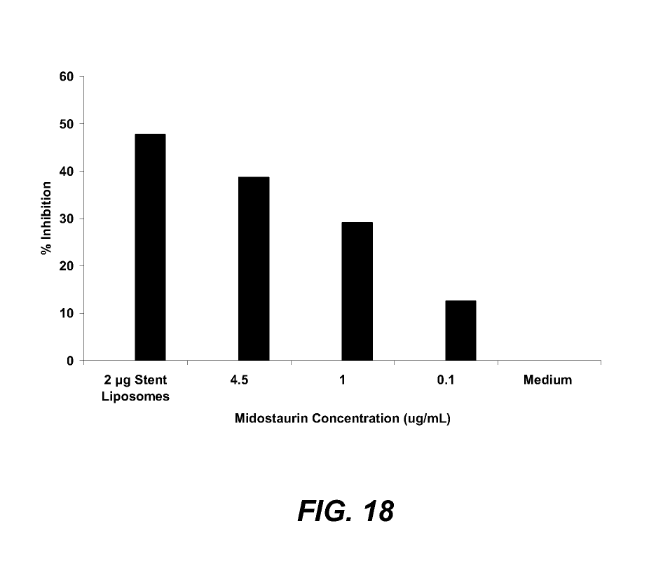

FIG. 18 is a plot of % inhibition of THP-1 cell growth (y-axis) for the various samples. It can be seen that that midostaurin released from the stent upon exposure to the culture medium (a 2 μg dose in 1 mL of medium), produced a slightly higher inhibitory effect on the cell growth as compared to 4.5 and 1 μg/mL concentrations of midostaurin in free molecular form.

Midostaurin is known to be a potent inhibitor of vascular smooth muscle cells on par with paclitaxel. Although it possesses some anti-inflammatory properties, this Example demonstrates that its anti-inflammatory potency can be improved through incorporation in a lipid bilayer film.

### Example 19

This Example describes the preparation of a coating capable of dual drug delivery for a single drug type.

A solution of castor oil and PKC-412 in DMSO is prepared where castor oil and PKC 412 have a weight ratio of 1.5:1 and the solution has a solid content of 5%. The above solution is sprayed on a surface of a hydroxyapatite coated stent. The sprayed stents are further placed under vacuum (30 mmHg) for 12 hours. The vacuum-treated stents are then spray coated with the formulation of Example 12. The stents are further placed under vacuum (30 mmHg) to remove the solvents. Within 48 hours of implantation, the bilayer film releases midostaurin in the form of liposomes. The midostaurin in the castor oil is released in molecular form for a longer period of time e.g. 30 days.

### Example 20

This Example demonstrates the effect of midostaurin formulated with castor oil contained on an HAp coated stent on in vitro growth of human coronary artery smooth muscle cells (HCASMC).

HCASMC were plated at 5×10⁴ cells/well into 12-well cluster plates (Corning) and cultured overnight in 1 mL of HCASMC growth medium. After 24 hours of incubation, 1 mL of medium in each well was replaced with 3 mL of fresh HCASMC growth medium. Stents with an HAp layer coated with castor oil with 10 μg of midostaurin were inserted into cell cultures. Wells with medium alone served as a background control. After one week of incubation, the cells in the cultures were harvested by trypsinization. The number of viable cells in each well was counted using a hemocytometer and trypan blue. The new cell cultures were started and stents were transferred into these new cultures for second, third, fourth etc. week evaluation.

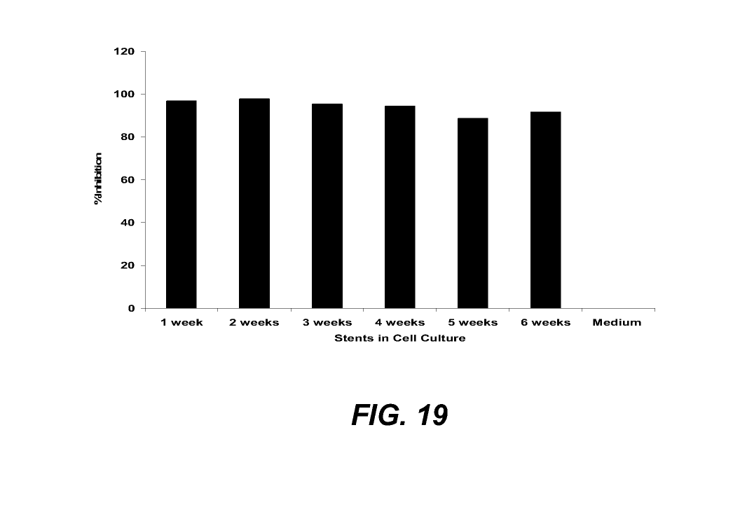

As presented in FIG. 19, which is a graph of % inhibition (y-axis) at various time intervals (weeks, x-axis),10 μg of midostaurin formulated with castor oil on an HAp coated stent was a very potent inhibitor of in vitro growth of HCASMC for six weeks.

This Example demonstrates that a non-liposome forming composition can function as an inhibitor of smooth muscle cell proliferation.

### REFERENCES

Allen T M, Austin G A, Chonn A, Lin L, Lee K C, 1991. Uptake of liposomes by cultured mouse bone marrow macrophages: influence of liposome composition and size. Biochim Biophys Acta 1061: 56-64.); Chono S, Tamino T, Seki T, Morimoto K, 2006. Influence of particle size on drug delivery to rat alveolar macrophages following pulmonary administration of ciprofloxacin incorporated into liposomes. J Drug Target 14(8): 57-66; Fidler I J, 1988. Targeting of immunomodulators to mononuclear phagocytes for therapy of cancer. Adv Drug Del Rev 2: 69-106; Huong T M, Harashima H, Kiwada H, 1998. Complement dependent and independent liposome uptake by peritoneal macrophages: cholesterol content dependency. Biol Pharm Bull 21(9): 969-973; Lee K D, Hong K, Papahadjopoulos D, 1992. Recognition of liposomes by cells: in vitro binding and endocytosis mediated by specific lipid headgroups and surface charge density. Biochim Biophys Acta 1103(2): 185-197; Pezzatini S, Solito R, Morbidelli L, Lamponi S, Boanini E, Bigi A, Ziche M. 2006. The effect of hydroxyapatite nanocrystals on microvascular endothelial cell viability and function. J Biomed Mater Res 76A: 656-663; Tsui M, 2007. Calcium phosphate coatings on coronary stents by electrophoretic deposition. M.A.Sc. Thesis, Department of Materials Engineering, University of British Columbia, Vancouver, BC.

A number of modifications and variations will readily suggest themselves to persons of ordinary skill in the art in view of the foregoing description. Directional words such as top, bottom, upper, lower, radial, circumferential, lateral, longitudinal and the like are employed by way of description and not limitation. The invention is intended to embrace all modifications and variations that fall within the scope of the appended claims.

1. A stent, comprising at least one coating covering at least a portion thereof, the at least one coating comprising a dry film comprising at least one lipid bilayer and at least one pharmaceutically effective agent.

2. The stent of claim 1, wherein the at least one coating is free of a polymer.

3. The stent of claim 1, wherein the at least one lipid bilayer comprises at least one lipid capable of forming a liposome encapsulating the pharmaceutically effective agent when exposed to an aqueous solution.

4. The stent of claim 1, wherein the at least one lipid bilayer comprises at least one lipid capable of forming a liposome encapsulating the pharmaceutically effective agent when released from the stent.

5. The stent of claim 1, wherein the coating further comprises a substrate covering at least a portion of the stent, and the dry film covers at least a portion of the substrate.

6. The stent of claim 5, wherein the substrate comprises a ceramic.

7. The stent of claim 6, wherein the ceramic is selected from at least one metal oxide and at least one calcium phosphate.

8. The stent of claim 7, wherein the at least one calcium phosphate comprises a hydroxyapatite.

9. The stent of claim 8, wherein the hydroxyapatite is a porous hydroxyapatite.

10. The stent of claim 5, wherein the substrate has a thickness of no more than 2 μm.

11. The stent of claim 5, wherein the substrate has a thickness of no more than 1 μm.

12. The stent of claim 5, wherein the substrate is porous.

13. The stent of claim 1, wherein the at least one pharmaceutically active agent is chosen from anti-inflammatory agents, anti-proliferatives, pro-healing agents, gene therapy agents, extracellular matrix modulators, anti-thrombotic agents, anti-platelet agents, antineoplastic agents, antisense agents, anti-angiogenic agents, anticoagulants, and antibiotics.

14. The stent of claim 1, wherein the at least one pharmaceutically active agent inhibits restenosis.

15. The stent of claim 1, wherein the at least one pharmaceutically active agent is selected from smooth muscle cell inhibitors, immunosuppressive agents, and anti-angiogenic agents.

16. The stent of claim 1, wherein the at least one pharmaceutically active agent is selected from sirolimus, paclitaxel, tacrolimus, heparin, pimecrolimus, imatinib mesylate, gentamycin, midostaurin, and bisphosphonates.

17. The stent of claim 16, wherein the bisphosphonates are selected from etidronate, clodronate, pamidronate, alendronate, risedronate, tiludronate, ibandronate, zoledronate, incadronate, olpadronate, neridronate, minodronate, YH 529, and EB-1053.

18. The stent of claim 1, wherein the film further comprises at least one anticoagulant.

19. The stent of claim 1, wherein the at least one lipid is selected from phospholipids and glycolipids.

20. The stent of claim 19, wherein the phospholipids are selected from phosphoglycerides.

21. The stent of claim 20, wherein the phosphoglycerides are selected from phosphatidylcholines, phosphatidylethanolamines, phosphatidylglycerols, and phosphatidic acids, phosphatidylserines, and phosphatidylinositols.

22. The stent of claim 21, wherein the phosphatidylcholines are selected from 1,2-dilauroyl-sn-glycero-3-phosphocholine, 1,2-dimyristoyl-sn-glycero-3-phosphocholine, 1,2-dipalmitoyl-sn-glycero-3-phosphocholine, 1,2-distearoyl-sn-glycero-3-phosphocholine, 1,2-dioleoyl-sn-glycero-3-phosphcholine, 1-palmitoyl-2-oleoyl-sn-glycero-3-phosphocholine, egg phosphatidylcholine, hydrogenated egg phosphatidylcholine, soybean phosphatidylcholine, and hydrogenated soybean phosphatidylcholine.

23. The stent of claim 21, wherein the phosphatidylethanolamines are selected from 1,2-dimyristoyl-sn-glycero-3-phosphoethanolamine, 1,2-dipalmitoyl-sn-glycero-3-phosphoethanolamine, 1,2-distearoyl-sn-glycero-3-phosphoethanolamine, and 1,2-dioleoyl-sn-glycero-3-phosphoethanolamine.

24. The stent of claim 21, wherein the phosphatidylglycerols are selected from egg phosphatidylglycerol, 1,2-dimyristoyl-sn-glycero-3-phosphoglycerol, 1,2-dipalmitoyl-sn-glycero-3-phosphoglycerol, 1,2-distearoyl-sn-glycero-3-phosphoglycerol, and 1-palmitoyl-2-oleoyl-sn-glycero-3-phosphoglycerol.

25. The stent of claim 21, wherein the phosphatidic acids are selected from 1,2-dimyristoyl-sn-glycero-3-phosphate, 1,2-dipalmitoyl-sn-glycero-3-phosphate, and 1,2-distearol-sn-glycero-3-phosphate.

26. The stent of claim 1, wherein the at least one lipid comprises a mixture of at least two lipids.

27. The stent of claim 26, wherein at least one of the at least two lipids is selected from cholesterol and derivatives thereof.

28. The stent of claim 27, wherein the cholesterol is selected from 7β-hydroxycholesterol 7-ketocholesterol, 7-ketocholesteryl acetate, 25-hydroxycholesterol, 24,25-epoxycholesterol, diacetylenic cholesterol, cholest-4-ene-3,6-dione, cholest-4-en-3-one, cholesteryl behenate, cholesteryl benzoate, cholesteryl butyrate, cholesteryl caprate, cholesteryl caproate, cholesteryl caprylate, cholesteryl-3,5-dinitrobenzoate, cholesteryl formate, cholesteryl-β-D-glucoside, cholesteryl hemisuccinate, cholesteryl heptylate, cholesteryl heptadecanoate, cholesteryl hydrogen phthalate, cholesteryl isobutyrate, cholesteryl isovalerate, cholesteryl laurate, cholesteryl linoleate, cholesteryl methyl succinates, cholesteryl myristate, cholesteryl nervonate, cholesteryl-p-nitrobenzoate, cholesteryl oleate, cholesteryl oleyl carbonate, cholesteryl palmitate, cholesteryl palmitelaidate, cholesteryl palmitoleate, cholesteryl phosphoryl choline, cholesteryl polyethylene glycols, cholesteryl propionate, cholesteryl N-propyl carbonate, cholesteryl 1-pyreecarbonate, cholesteryl (pyren-1-yl) hexanoate, cholesteryl stearate, cholesteryl-P-tosylate, cholesteryl valerate, and thiocholesterolcholesteryl sulfate.

29. The stent of claim 26, wherein the film comprises a phosphatidylcholine and a cholesterol or derivatives thereof.

30. The stent of claim 1, wherein the film further comprises at least one anticoagulant.

31. A method of preparing a coating for a stent, comprising:
combining at least one lipid with at least one pharmaceutically active agent to form a composition comprising at least one lipid bilayer; and
coating at least a portion of the stent with the composition.

32. The method of claim 31, further comprising depositing a substrate on at least a portion of the stent, and the coating of the composition is performed on at least a portion of the substrate.

33. The method of claim 32, wherein the depositing comprises depositing the substrate from a solution or suspension.

34. The method of claim 32, wherein the depositing comprises at least one method selected from electrochemical deposition, electrophoretic deposition (EPD), sol gel processes, aero-sol gel processes, biomimetic (BM) processes, spraying, and dipping.

35. A method of preparing a coating for an implantable medical device, comprising:
coating at least a portion of the device with a substrate by at least one method selected from electrochemical deposition, electrophoretic deposition (EPD), sol gel processes, aero-sol gel processes, biomimetic (BM) processes, spraying, and dipping;
combining at least one lipid with at least one pharmaceutically active agent to form a composition comprising at least one lipid bilayer; and
coating at least a portion of the substrate with the composition.

36. The method of claim 35, wherein the coating step comprises electrochemical deposition.

37. The method of claim 35, wherein the combining comprises forming a solution comprising the at least one lipid with at least one pharmaceutically active agent.

38. The method of claim 37, wherein the at least one pharmaceutically active agent is hydrophobic or amphipathic.

39. The method of claim 35, wherein the combining comprises forming a water-in-oil emulsion comprising the pharmaceutically active agent in the aqueous phase within a non-aqueous phase comprising the at least one lipid.

40. The method of claim 39, wherein the at least one pharmaceutically active agent is hydrophilic or amphipathic.

41. The method of claim 39, wherein the emulsion further comprises at least one additional surfactant.

42. The method of claim 41, wherein the at least one additional surfactant is selected from chitosan, didodecyldimethylammonium bromide, dextrans, dextran salts, polyoxyethylene castor oil, polyoxyethylene 35 soybean glycerides, glyceryl monooleate, triglyceryl monoleate, glyceryl monocaprylate, glycerol monocaprylocaprate, propylene glycol monolaurate, triglycerol monooleate, stearic glycerides, sorbitane monostearate, sorbitan monooleate, polyoxyethylene sorbitan monolaurate, polyoxyethylenesorbitan tristearate, and polyoxyethylene sorbitan monooleate.

43. A method of preparing a coating for an implantable medical device, comprising:
coating at least a portion of the device by depositing a substrate from a solution or suspension;
combining at least one lipid with at least one pharmaceutically active agent to form a composition comprising at least one lipid bilayer; and
coating at least a portion of the substrate with the composition.

44. A method of treating at least one disease or condition comprising:
implanting in a subject in need thereof a medical device comprising a coating covering at least a portion of the device, the coating comprising at least one lipid bilayer and a therapeutically effective amount of at least one pharmaceutically active agent, and
releasing from the device the at least one pharmaceutically active agent encapsulated in a liposome comprising lipids from the lipid bilayer.

45. The method of claim 44, wherein the releasing comprises contacting the coating with an aqueous or physiological medium.

46. The method of claim 44, wherein the device is implanted in a mammalian lumen.

47. The method of claim 46, wherein the at least one disease or condition is associated with narrowing or obstruction of the mammalian lumen.

48. The method of claim 44, wherein the at least one disease or condition is a proliferative disorder.

49. The method of claim 48, wherein the proliferative disorder is restenosis.

50. The method of claim 48, wherein the proliferative disorder is a tumor.

51. The method of claim 48, wherein the proliferative disorder comprises the proliferation of smooth muscle cells.

52. The method of claim 44, wherein the at least one disease or condition is an inflammatory disease.

53. The method of claim 44, wherein the at least one disease or condition is thrombosis.

54. A medical device comprising at least one coating covering at least a portion of the device, the at least one coating comprising:
a porous substrate;
a composition contacting the porous substrate, the composition comprising at least one lipid and at least one pharmaceutically effective agent wherein the at least one lipid does not form a lipid bilayer film; and
a dry lipid bilayer film contacting the porous substrate and/or the composition, the dry film comprising at least one pharmaceutically effective agent that can be the same or different from the agent in the composition.

55. The device of claim 54, wherein the at least one pharmaceutically effective agent in the composition acts primarily as an anti-proliferative agent and the agent in the dry bilayer film acts primarily as an anti-inflammatory agent.

56. A medical device comprising at least one coating covering at least a portion of the device, the at least one coating comprising:
a porous substrate;
a composition impregnating the porous substrate, the composition comprising at least one lipid and at least one pharmaceutically effective agent;
a film overcoating the composition, the film comprising at least one pharmaceutically effective agent and at least one lipid.

57. The device of claim 56, wherein the film comprises the at least one lipid in the form of a lipid bilayer.

58. The device of claim 56, wherein the at least one pharmaceutically effective agent in the composition is the same as the agent in the film.

59. The device of claim 58, wherein the at least one pharmaceutically effective agent in the composition acts primarily as an anti-proliferative agent and the agent in the film acts primarily as an anti-inflammatory agent.

60. The device of claim 56, wherein the at least one pharmaceutically effective agent in the composition is the different from the agent in the film.

61. A medical device comprising at least one coating covering at least a portion of the device, the at least one coating comprising:
a porous substrate;
at least one pharmaceutically effective agent impregnating the porous substrate; and
a film overcoating the porous substrate, the film comprising at least one pharmaceutically effective agent and at least one lipid.

62. The device of claim 61, wherein the film comprises the at least one lipid in the form of a lipid bilayer.

63. The device of claim 61, wherein the at least one pharmaceutically effective agent impregnating the porous substrate is the same as the agent in the film.

64. The device of claim 61, wherein the at least one pharmaceutically effective agent impregnating the porous substrate acts primarily as an anti-proliferative agent and the agent in the film acts primarily as an anti-inflammatory agent.

65. The device of claim 61, wherein the at least one pharmaceutically effective agent in the composition is the different from the agent in the layer.

66. A medical device comprising at least one coating covering at least a portion of the device, the at least one coating comprising:
a porous substrate;
a film deposited on the substrate comprising at least one lipid and at least one pharmaceutically active agent; and
at least one pharmaceutically active agent contacting the porous substrate and free of contact with the film.

67. The device of claim 66, wherein the porous substrate is a ceramic.

68. The device of claim 66, wherein the at least one pharmaceutically effective agent contacting the ceramic is primarily an anti-proliferative agent and the agent in the film is primarily an anti-inflammatory agent.

69. The device of claim 66, wherein the at least one coating is free of a polymer.

70. The device of claim 66, wherein the ceramic is bioresorbable and releases the at least one pharmaceutically active agent contacting the ceramic upon resorption of the ceramic.

71. A method of treating at least one disease or condition comprising:
implanting in a subject in need thereof a medical device comprising at least one coating covering at least a portion of the device, the at least one coating comprising:
a composition comprising at least one lipid and at least one pharmaceutically active agent, the composition covering at least a portion of the device; and
releasing from the device the least one pharmaceutically active agent encapsulated in the at least one lipid.

72. The method of claim 71, wherein the at least one coating further comprises a substrate and the composition covers at least a portion of the substrate.

73. The method of claim 71, wherein the at least one lipid is present as a lipid bilayer, and the at least one agent is encapsulated in the liposome.

74. The method of claim 73, wherein the liposomes have a particle size distribution, wherein 25% or less of the particles have an average diameter of less than 1 μm, and at least 25% of the particles have an average diameter of greater than 1 μm.

75. The method of claim 74, wherein at least 50% of the particles have an average diameter of greater than 2 μm.

76. The method of claim 74, wherein the liposomes having a particle size greater than 1 μm are anti-inflammatory agents.

77. The method of claim 74, wherein the liposomes having a particle size less than 1 μm and any drug released in molecular form are anti-proliferative agents.

78. The method of claim 71, wherein the at least one disease or condition is associated with restenosis.
